SEQUENCE LISTING (6 sequences)

SEQ ID NO: 1 - protein, 226 aa, Hepatitis B virus
MENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 2 - protein, 412 aa, Artificial
  ZZ-BNC
KVRQGMGTNLSVPNPLGFFPDHQLDPAFGANSNNPDWDFNPNKDQWPEANQVGAGGRAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKAAAPAPNMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 3 - protein, 59 aa, Artificial Sequence
  Antibody binding domain in Protein A
DVDNKFNKEQQNAFWEILHLPNLNEEQRNGFIQSLKDDPSQSANLLAEAKKLNDAQAPK

SEQ ID NO: 4 - protein, 55 aa, Artificial Sequence
  Antibody binding domain in Protein G
TYKLVINGKTLKGETTTEAVDAATAEKVFKQYANDNGVDGEWTYDDATKTFTVTE

SEQ ID NO: 5 - protein, 81 aa, Artificial Sequence
  Antibody binding domain in Protein L
PFVENKEETPETPETDSEEEVTIKANLIFANGSTQTAEFKGTFEKATSEAYAYADTLKKDNGEYTVDVADKGYTLNIKFAG

SEQ ID NO: 6 - protein, 223 aa, Artificial Sequence
  AGG peptide described in example
MGHHHHHHHHHHSSGHIDDDDKHMLEDVDNKFNKEQQNAFWEILHLPNLNEEQRNGFIQSLKDDPSQSANLLAEAKKLNDAQAPKGGGGSTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEGSHHHHHH

(19) United States
(12) Patent Application Publication
(10) Pub. No.: US 2016/0202251 A1
(43) Pub. Date: Jul. 14, 2016

(54) VIRUS-LIKE PARTICLE FOR USE IN IMMUNOASSAY, BLOCKING AGENT FOR USE IN THE IMMUNOASSAY, AND KIT COMPRISING THE VIRUS-LIKE PARTICLE AND THE BLOCKING AGENT

(71) Applicant: BEACLE INC., Kyoto (JP)

(72) Inventors: Yasumasa GOH, Kyoto (JP); Yasunori ODA, Kyoto (JP)

(73) Assignee: BEACLE INC., Kyoto (JP)

(21) Appl. No.: 14/897,274

(22) PCT Filed: Aug. 1, 2014
(86) PCT No.: PCT/JP2014/070396

§ 371 Date: Dec. 10, 2015

(51) Int. Cl. G01N 33/543; G01N 33/544; C07K 14/195; C07K 14/31; C07K 14/315; G01N 33/569; C07K 14/005

(52) U.S. Cl. CPC G01N 33/54346; G01N 33/56983; G01N 33/56905; G01N 33/56966; G01N 33/544; C07K 14/005; C07K 14/31; C07K 14/315; C07K 14/195; G01N 2333/02; G01N 2333/44; G01N 2333/71; C12N 2730/10123

(57) ABSTRACT

An object of the present invention is to provide an immunoassay that has excellent detection sensitivity and that remarkably suppresses the detection background. The means for achieving the object is to provide a virus-like particle that contains a protein having self-organization ability, the particle being modified with a biologically active molecule at at least one cysteine residue of the protein having self-organization ability via a thiol group or thiol groups thereof.

### TECHNICAL FIELD

The present invention relates to a virus-like particle for use in an immunoassay, a blocking agent for use in the immunoassay, and a kit comprising the virus-like particle and the blocking agent.

### BACKGROUND ART

Methods for using various particles as immunoassay sensors are known (PTL 1 to 8). For example, PTL 1 or PTL 2 discloses a method for utilizing fluorescent semiconductor nanoparticles or silica particles as immunoassay detection elements, which is a method for performing an immunoassay using fluorescence emitted by nanoparticles.

PTL 8 discloses a nanoparticle conjugate comprising a metallic material, a magnetic material, or a semiconductor material as the core and various peptides conjugated thereto.

A virus-like particle having a hepatitis B virus surface antigen that has two protein A-derived binding domains for the FC region of an antibody (Z tags) inserted in tandem into the Pre-S1 and Pre-S2 regions at the N terminus of the antigen (hereinafter sometimes referred to as “ZZ-BNC”) is a particle in which two protein A-derived binding domains (Z tags) that bind to the Fc region of an antigen are inserted into the Pre-S1 and Pre-S2 regions of an L hepatitis B virus particle recombinantly produced using yeast (PTL 9). PTL 10 discloses a method for producing this particle and the usefulness of this particle as a drug delivery system.

Focusing attention on the high function of this BNC-ZZ as a sensor element, the Examples of PTL 3 disclose a method for using a combination of an enzyme-labeled antibody and BNC-ZZ, a method for detecting an antibody using fluorescently labeled BNC-ZZ, and a method for utilizing BNC-ZZ in an immunoassay. PTL 6 discloses a method for enhancing sensitivity by using BNC-ZZ to array immobilized antibodies. PTL 4 discloses a method for applying biotinylated BNC-ZZ to an immunoassay and shows in the Examples that biotinylated BNC-ZZ to which a biotinylated HRP enzyme or a biotinylated antibody is bound via streptavidin has enhanced sensitivity, compared to unbiotinylated BNC-ZZ.

PTL 5 discloses a method for producing and utilizing a hybrid particle comprising a protein molecule not having a Pre-S region and a protein molecule having a ZZ tag in order to increase the sensitivity of an immunoassay by increasing the antibody-binding capacity of BNC-ZZ particles. Further, the Examples of PTL 7 disclose a method for simultaneously detecting multiple antigens by weakly crosslinking antibodies to fluorescently labeled BNC-ZZ.

### CITATION LIST

### Patent Literature

PTL 1: JP2006-517985A
PTL 2: JP2002-544488A
PTL 3: JP2007-127626A
PTL 4: JP2008-191143A
PTL 5: JP2010-096677A
PTL 6: JP2007-121276A
PTL 7: JP2010-210444A
PTL 8: JP2007-506084A
PTL 9: JP2001-316298A
PTL 10: JP2004-002313A

### Non-Patent Literature

NPL 1: Vaccine 19-3154-3163, 2001

### SUMMARY OF INVENTION

### Technical Problem

In general, a method for enhancing sensitivity in an immunoassay is either (1) using a detection element that has a high affinity for the substance to be detected by the element, or (2) increasing the intensity of the signal generated by a detection element bound to a substance to be detected. When BNC-ZZ as described above is used as a detection element, its affinity for the substance to be detected is constant. Therefore, enhancing the intensity of the generated signal is the most effective means.

However, PTL 4, a document that discloses that BNC-ZZ was utilized, only discloses a method for using biotinylated BNC-ZZ as a method for increasing the signal by utilizing a well-known specific binding capacity between biotin and streptavidin to increase the amount of HRP bound, and no study is conducted on BNC-ZZ.

ZZ-BNC, which has protein A-derived antibody binding sites, has weak binding to antibodies important in an immunoassay, such as mouse IgG₁, rat IgG, sheep IgG₁, goat IgG₁, and human IgG₃, and the method and range of its use are severely limited. Furthermore, since ZZ-BNC is bound to various IgGs, binding to antibodies other than the target substance occurs in an environment in which multiple antibodies are present, such as an antibody-sandwich ELISA, or an evaluation of serum or the like in which multiple antibodies are present; therefore, an antibody-specific detection is difficult.

When practical application is considered, BNC-ZZ, labeled BNC-ZZ, and modified BNC-ZZ are lipoproteins and thus nonspecifically bind to glass and plastics. Non-specific adsorption not only greatly affects the immunoassay but also poses a serious problem in the long-term storage of diluted solutions.

### Solution to Problem

The present inventors carried out extensive research to solve the above problem. As a result, the inventors found that when a virus-like particle is labeled with a biologically active molecule via a specific site of a protein having self-organization ability contained in the virus-like particle, the resulting virus-like particle can be suitably used for immunoassays.

The present inventors further found that in immunoassays using this virus-like particle, a specific blocking agent provides excellent blocking effects.

The present invention has been accomplished based on the above findings, and includes the following broad aspects.

Item 1 A virus-like particle for an immunoassay containing a protein having self-organization ability, the particle being modified with a biologically active molecule at at least one cysteine residue of the protein via a thiol group or thiol groups thereof.
Item 2 The virus-like particle according to Item 1, wherein the protein having self-organization ability is an HBsAg protein.
Item 3 The virus-like particle according to Item 1, wherein the protein having self-organization ability comprises an amino acid sequence of SEQ ID NO: 1.
Item 4 The virus-like particle according to any one of Items 1 to 3, wherein the protein having self-organization ability has an antibody-binding domain.
Item 5 The virus-like particle according to any one of Items 1 to 4, wherein the biologically active molecule is at least one member selected from the group consisting of an enzyme, an antibody-binding domain, biotin, a fluorescent dye, a luminescent dye, and an avidin compound.
Item 6 The virus-like particle according to Item 5, wherein the enzyme is alkaline phosphatase and/or peroxidase.
Item 7 The virus-like particle according to Item 4 or 5, wherein the antibody-binding domain is at least one member selected from the group consisting of antibody-binding domains of protein A, antibody-binding domains of protein G, and antibody-binding domains of protein L.
Item 8 The viral particle according to Item 4 or 5, wherein the antibody-binding domain consists of an amino acid sequence of any one of SEQ ID NOS: 3 to 5.
Item 9 The virus-like particle according to Item 5, wherein the avidin compound is at least one member selected from the group consisting of avidin, streptavidin, neutravidin, AVR protein, Bradavidin, Rhizavidin, and Tamavidin®.
Item 10 The virus-like particle according to any one of Items 1 to 9, wherein the biologically active molecule is an antibody-binding domain, and wherein an antibody is bound to the antibody-binding domain.
Item 11 A blocking agent for an immunoassay using the virus-like particle according to any one of Items 1 to 10, the blocking agent containing at least one member selected from the group consisting of hydroxyalkyl cellulose, polyvinyl alcohol, an ethylene oxide-propylene oxide copolymer, and a copolymer of 2-methacryloyloxyethylphosphocholine.
Item 12 The blocking agent according to Item 11, wherein the hydroxyalkyl cellulose is hydroxypropyl methylcellulose.
Item 13 The blocking agent according to Item 11, wherein the polyvinyl alcohol has a degree of polymerization of 200 to 5,000.
Item 14 The blocking agent according to Item 11, wherein the ethylene oxide-propylene oxide copolymer is Pluronic®.
Item 15 The blocking agent according to Item 11, wherein the ethylene oxide-propylene oxide copolymer is Pluronic® F127 and/or Pluronic® P105.
Item 16 The blocking agent according to Item 11, wherein the copolymer of 2-methacryloyloxyethylphosphocholine is Biolipidure®.
Item 17 The blocking agent according to Item 11, wherein the copolymer of 2-methacryloyloxyethylphosphocholine is Biolipidure® 206 and/or Biolipidure® 802.
Item 18 A kit for an immunoassay comprising the virus-like particle according to any one of Items 1 to 10 and the blocking agent according to any one of Items 11 to 17.

### Advantageous Effects of Invention

The virus-like particle according to the present invention enables an immunoassay with excellent detection sensitivity.

In immunoassays using the virus-like particle of the present invention, the blocking agent according to the present invention can function to reduce the background in the data obtained and/or sensitize the detection signal, thus remarkably increasing the S/N ratio.

The immunoassay using the kit of the present invention can remarkably increase the S/N ratio of the obtained data.

### BRIEF DESCRIPTION OF DRAWINGS

FIG. 1 shows the results of Example 5.

FIG. 2 shows the results of Example 6.

FIG. 3 shows the results of Example 16.

FIG. 4 shows the results of Example 17.

FIG. 5 shows the results of Example 18.

FIG. 6 shows the results of Example 20.

FIG. 7 shows the results of Example 21.

FIG. 8 shows the results of Example 22.

FIG. 9 shows the results of Example 23.

FIG. 10 shows the results of Example 24 (anti-GFP antibody).

FIG. 11 shows the results of Example 24 (HMG monoclonal antibody).

FIG. 12 shows the results of Example 25.

FIG. 13 shows the results of Example 27.

FIG. 14 shows the results of Example 28.

FIG. 15 shows the results of Example 29.

FIG. 16 shows the results of Example 30.

FIG. 17 shows the results of Example 31.

FIG. 18 shows the results of Example 32.

FIG. 19 shows the results of Example 33.

FIG. 20 shows the results of Example 34.

FIG. 21 shows the results of Example 35.

FIG. 22 shows the results of Example 36.

### Virus-Like Particle

The virus-like particle according to the present invention is used in immunoassays and contains a protein having self-organization ability. The protein having self-organization ability is modified with a biologically active molecule at at least one cysteine residue of the protein via a thiol group or thiol groups thereof.

The protein having self-organization ability refers to a protein capable of forming a virus-like particle by enclosing a lipid bilayer membrane, such as an endoplasmic reticulum lumen, cell membrane, or nuclear membrane, in vivo, in particular, in cells, and is not particularly limited as long as the protein has a cysteine residue. Examples of such proteins include proteins involved in the budding function of viruses having envelopes, envelope proteins, variants of these proteins, and the like.

The viruses having envelopes are not particularly limited. Examples of such viruses include viruses that belong to the Hepadnaviridae family, such as Hepatitis B virus (HBV) and Duck hepatitis B virus; viruses that belong to the Paramyxoviridae family, such as Sendai virus (HVJ); viruses that belong to the Herpesviridae family, such as herpes simplex viruses; viruses that belong to the Orthomyxoviridae family, such as influenza viruses; viruses that belong to the Retroviridae family, such as human immunodeficiency viruses; and the like.

Examples of proteins having self-organization ability include, but are not limited to, a hepatitis B virus surface antigen (HBsAg) protein, which is a protein involved in the budding function of HVB, protein F, which is a protein involved in the budding function of HVJ, hemagglutinin neuraminidase protein, and variants of these proteins. Among these, HBsAg protein, protein F, hemagglutinin neuraminidase protein, variants of these proteins, and the like are preferable.

The variant is not particularly limited as long as it has at least one cysteine residue and it functions to form a virus-like particle as described above. The specific number of mutations introduced is also not particularly limited as long as the resulting variant satisfies the above conditions. The number of mutations introduced may be typically such that the resulting variant has 85% or higher identity, preferably 90% or higher identity, more preferably 95% or higher identity, and most preferably 99% or higher identity with the amino acid sequence before mutation.

The term “mutation” as used herein includes substitution, deletion, insertion, and the like. As a specific method for introducing mutations, known methods can be used without any specific limitation. For example, to introduce a substitution, a conservative substitution technique may be employed. The term “conservative substitution technique” means the substitution of an amino acid residue with another amino acid residue that has a similar side chain.

The conservative substitution technique includes, for example, a substitution between amino acid residues having basic side chains, such as lysine, arginine, and histidine. Other examples of the conservative substitution technique include substitutions between amino acid residues having acidic side chains, such as aspartic acid and glutamic acid; substitutions between amino acid residues having uncharged polar side chains, such as glycine, asparagine, glutamine, serine, threonine, tyrosine, and cysteine; substitutions between amino acid residues having non-polar side chains, such as alanine, valine, leucine, isoleucine, proline, phenylalanine, methionine, and tryptophan; substitutions between amino acid residues having β-branched side chains, such as threonine, valine, and isoleucine; and substitutions between amino acid residues having aromatic side chains, such as tyrosine, phenylalanine, tryptophan, and histidine.

The term “identity” refers to the degree of identical amino acid sequences between two or more comparable amino acid sequences. Accordingly, when the identity between two amino acid sequences or nucleotide sequences is high, the identity or similarity of these sequences is high. The level of identity between amino acid sequences is determined, for example, using FASTA, which is a sequence analysis tool, based on default parameters. Specific techniques of these analysis methods are known. Reference can be made to the website of the National Center of Biotechnology Information (NCBI) (http://www.ncbi.nlm.nih.gov/).

Examples of HBsAg include, but are not limited to, proteins comprising the amino acid sequence of SEQ ID NO: 1. Examples of HBsAg variants include HBsAg variants disclosed in PTL 5 and 9, and the like. Examples of Protein F include, but are not limited to, proteins consisting of the amino acid sequence described under Accession No. NP_056877; Version: NP_056877.1 GI: 9627226 registered at the website of NCBI. Examples of the hemagglutinin neuraminidase protein include, but are not limited to, proteins consisting of the amino acid sequence described under Accession No. NP_056878; Version: NP_056878.1 GI: 9627227 registered at the website of NCBI.

The protein having self-organization ability has cysteine residues and is modified with a biologically active molecule via one or more thiol groups of the cysteine residues. The cysteine residue is not limited but is preferably a specific cysteine residue that is located on the surface of a virus-like particle formed on the basis of the protein having self-organization ability and that is modified with a biologically active molecule.

Such a cysteine residue of, for example, the HBsAg protein comprising the amino acid sequence of SEQ ID NO: 1 may be a cysteine residue at position 107, 121, 124, 137, 138, 139, 147, or 149. As transmembrane domains of the HBsAg protein, the amino acid sequences represented by amino acid numbers 9 to 28, 80 to 98, and 170 to 192 are deduced.

Such a cysteine residue of, for example, protein F consisting of the amino acid sequence shown at the website of NCBI may be a cysteine residue at position 70, 199, 338, 347, 362, 370, 394, 399, 401, or 424. As transmembrane domains of protein F, the amino acid sequences represented by amino acid numbers 1 to 25, 117 to 139, and 501 to 523 are deduced.

Such a cysteine residue of, for example, a hemagglutinin neuraminidase protein consisting of the amino acid sequence shown at the website of NCBI may be a cysteine residue at position 129, 138, 161, 192, 216, 258, 271, 352, 357, 365, 463, 469, 473, 535, 544, or 571. As a transmembrane domain of the hemagglutinin neuraminidase protein, the amino acid sequence represented by amino acid numbers 38 to 60 is deduced.

Examples of biologically active molecules include, but are not limited to, enzymes, antibody-binding domains, biotin, fluorescent dyes, luminescent dyes, avidin compounds, and the like. As described above, the protein having self-organization ability of the present invention has one or more cysteine residues and is bound to a biologically active molecule via at least one of the cysteine residues. Accordingly, one or a combination of two or more of the above enzymes, antibody-binding domains, biotin, fluorescent dyes, luminescent dyes, avidin compounds, etc., may be bound to the protein via individual cysteine residues.

Specific examples of enzymes may be any enzyme commonly used in the field of immunoassays, and include peroxidase, alkaline phosphatase, and the like.

Specific examples of antibody-binding domains are not limited, and domains that bind to the Fc domains of antibodies are preferable. Examples of specific antibody-binding domains include, but are not limited to, an antibody-binding domain contained in protein A (SEQ ID NO: 3), an antibody-binding domain contained in Protein G (SEQ ID NO: 4), an antibody-binding domain contained in Protein L (SEQ ID NO: 5), and the like.

The antibody-binding domain included in the biologically active molecule may be an embodiment containing two or more such identical or different antibody-binding domains. Examples of such embodiments include an antibody-binding domain (SEQ ID NO: 6) comprising an amino acid sequence comprising, in order from the N-terminus, an antibody-binding domain of protein A, an antibody-binding domain of Protein G, and an antibody-binding domain of Protein G; and a ZZ tag in which antibody binding domains of protein A are inserted in tandem in order from the N-terminus; and the like.

The antibody that is bound to the antibody-binding domain is not particularly limited and not structurally limited to immunoglobulins. Any molecule that has a structure capable of at least recognizing antigens may be used. For example, antibodies that have a building block structure, such as multivalent antibodies, may be used.

The origin of the antibody is also not particularly limited. Antibodies derived from various animals suitable for producing antibodies can be used.

For example, when the antibody is an immunoglobulin, the subtype is not particularly limited. When the immunoglobulin is IgG, IgA, or the like, the subclass is also not particularly limited.

When the biologically active molecule is an antibody-binding domain, an embodiment in which the domain is bound to the above antibody is also included within the scope of the virus-like particle according to the present invention. The antibody-binding domain and the antibody may be strongly bound by using a known crosslinking agent (sometimes herein referred to as “irreversible binding”). Examples of such known crosslinking agents include BS₃ and the like.

Biotin, fluorescent dyes, and luminescent dyes are not particularly limited as long as they are commonly used in the field of immunoassays. Any known biotin, fluorescent dyes, and luminescent dyes may be appropriately used.

Specific examples of avidin compounds include, but are not limited to, avidin, streptavidin, neutravidin, AVR protein, bradavidin, rhizavidin, tamavidin, and the like.

The virus-like particle according to the present invention can be produced by obtaining a virus-like particle comprising a protein having self-organization ability using a known method, and binding a biologically active molecule as mentioned above to the obtained virus-like particle via a cysteine residue using a kit for modifying a protein or the like. After binding, the obtained virus-like particle may be subjected to purification appropriately using a known method, such as gel filtration.

After a biologically active molecule is bound to the virus-like particle, the resulting particle may be treated with a known crosslinking agent using a known method to strengthen the binding between the biologically active molecule and the cysteine residue of the protein having self-organization ability contained in the virus-like particle. Examples of known crosslinking agents include BS₃ and the like.

The virus-like particle according to the present invention encompasses an embodiment of a virus-like particle comprising a protein having self-organization ability that is bound to a biologically active molecule via the aforementioned cysteine residue and also to another biologically active molecule (hereinafter sometimes referred to as “a second biologically active molecule”).

Examples of such embodiments of virus-like particles include an embodiment in which a lipid component that forms a lipid bilayer membrane of the virus-like particle is bound to a second biologically active molecule, an embodiment in which a second biologically protein is bound via an amino acid other than the above-mentioned cysteine residue of the protein having self-organization ability or via a sugar chain, an embodiment in which a second biologically active molecule is incorporated into a specific site of the protein having self-organization ability.

The second biologically active molecule may be the same as the biologically active molecule modified via the above-mentioned cysteine residue.

An example of an embodiment in which an antibody-binding domain is incorporated into the N-terminal region of the protein having self-organization ability as a second biologically active molecule is a virus-like particle having a protein having self-organization ability comprising the amino acid sequence of SEQ ID NO: 2. Specifically, such a protein having self-organization ability may have an antibody-binding domain as a biologically active molecule bound via a thiol group of at least one of the cysteine residues of the protein and in the N-terminal region of this protein.

For example, an embodiment in which a second biologically active molecule is bound to a sugar chain of the protein having self-organization ability can be obtained by aldehydizing the terminal sugar residue of a sugar chain, such as sialic acid.

The binding of the protein having self-organization ability and the second biologically active molecule can be strengthened by using a known crosslinking agent as described above. Examples of such known crosslinking agents include BS₃ and the like.

When the second biologically active molecule is an antibody-binding domain, an embodiment in which an antibody is bound to the domain is also included within the scope of the virus-like particle according to the present invention. An embodiment in which the virus-like particle is subjected to crosslinking treatment to strengthen the binding as necessary is also included within the scope of the virus-like particle of the present invention. Examples of such known crosslinking agents include BS₃ and the like.

Preferred embodiments of the virus-like particle according to the present invention include virus-like particles that contain a protein having self-organization ability and that satisfy one of the following: (1) the virus-like particle has an antibody-binding domain in the N-terminal region of the protein and also has an enzyme bound thereto via the above-mentioned specific cysteine residue of the protein (corresponding to “SH-HRP-labeled BNC-ZZ” in Production Example 3 and “SH-ALP labeled BNC-ZZ” in Production Example 4 of the Examples below); (2) the virus-like particle has an antibody-binding domain in the N-terminal region of the protein and the antibody-binding domain is bound via the above-mentioned specific cysteine residue of the protein (corresponding to “AGG-BNC-ZZ” in Production Example 5 of the Examples below); (3) the virus-like particle has an antibody-binding domain in the N-terminal region of the protein, the antibody-binding domain is bound via the above-mentioned specific cysteine residue of the protein, and the virus-like particle has an enzyme bound via the N-terminus or lysine residue of the protein (corresponding to “HRP-labeled AGG-BNC-ZZ” in Production Example 6 of the Examples below; (4) the virus-like particle has an avidin compound bound via the above-mentioned specific cysteine residue of the protein (corresponding to “BNC-SA” in Production Example 8 of the Examples below); (5) the virus-like particle has an avidin compound bound via the above-mentioned specific cysteine residue of the protein and has an enzyme bound via a lysine residue of the protein (corresponding to “HRP-labeled BNC-SA” in Production Example 8 of the Examples below); (6) the virus-like particle has an enzyme bound via the above-mentioned specific cysteine residue of the protein (corresponding to “HRP-labeled BNC-L” in Production Example 9 of the Examples below); (7) the virus-like particle has an antibody-binding domain bound via a sugar chain of the protein and has an enzyme bound via the above-mentioned specific cysteine residue of the protein (corresponding to “SH-HRP-labeled BNC-(sugar chain)-AGG” in Production Example 10 of the Examples below; (8) the virus-like particle has an enzyme bound via the above-mentioned specific cysteine residue of the protein (corresponding to HRP-labeled HVJ-E in Production Example 11 of the Examples below); and the like.

The immunoassay as defined herein may be any measurement method using the antigen-antibody binding action of an antibody as a measurement principle. Examples of such immunoassays include Western blotting, ELISA, immunochromatography, immunostaining, EIA, FIA, and various modifications on the basis of these assays.

### Blocking Agent

The blocking agent according to the present invention is used in immunoassays using the virus-like particle of the present invention. This blocking agent functions to reduce the background of data obtained in the above immunoassay and/or sensitize the detection signal, thus remarkably increasing the S/N ratio.

These effects are evaluated not only based on the obtained data but also, for example, by ascertaining the inhibition of adsorption of the virus-like particle on laboratory instruments commonly used in an immunoassay.

The virus-like particle and immunoassay may be as described in detail in the above Virus-like Particle section.

The blocking agent according to the present invention contains hydroxyalkyl cellulose, polyvinyl alcohol, an ethylene oxide-propylene oxide copolymer, a copolymer of 2-methacryloyloxyethylphosphocholine, or two or more of these compounds.

The hydroxyalkyl cellulose is not particularly limited but preferably contains an alkyl group having about 1 to 4 carbon atoms. Examples of hydroxyalkyl celluloses include hydroxypropyl cellulose, hydroxyethyl cellulose, hydroxypropyl methyl cellulose, hydroxyethyl methyl cellulose, hydroxypropyl ethyl cellulose, hydroxyethyl ethyl cellulose, and the like. Among these, hydroxypropyl methyl cellulose is preferable.

The hydroxyalkyl cellulose contained in the blocking agent of the present invention may be a combination of two or more of the above compounds. Specific methods for obtaining hydroxyalkyl cellulose include purchasing from the market, production using known methods, and the like.

The polyvinyl alcohol may be any polymer having vinyl alcohol as a monomer unit. The degree of polymerization of the polyvinyl alcohol is not particularly limited, and is typically preferably about 200 to 5,000, and more preferably about 500 to 2,000.

Polyvinyl alcohols commonly used are often produced by saponifying polyvinyl acetate. Therefore, the polyvinyl alcohol may contain vinyl acetate as a monomer unit as described above. From this viewpoint, the polyvinyl alcohol may typically have a saponification degree (mol %) of about 80 to 98, and preferably about 85 to 98.

The polyvinyl alcohol contained in the blocking agent of the present invention may be a combination of two or more of the above compounds. Specific methods for obtaining such polyvinyl alcohols include purchasing from the market, production using known methods, and the like.

The ethylene oxide-propylene oxide copolymer may be any copolymer of ethylene oxide and propylene oxide as monomer units. For example, Pluronic® or equivalents thereof are preferable.

Examples of more preferable ethylene oxide-propylene oxide copolymers include Pluronic L31, Pluronic L35, Pluronic L64, Pluronic P94, Pluronic F68, Pluronic F87, Pluronic F-127, Pluronic P-105, and the like (all are trademarks of BASF). For example, an equivalent of these products is Poloxamer (trademark of ICI). Among these, Pluronic F-127, Pluronic P-105, equivalents thereof, and the like are the most preferable.

The ethylene oxide-propylene oxide copolymer contained in the blocking agent according to the present invention may be a combination of two or more of the above compounds. Specific methods for obtaining the blocking agent include purchasing from the market (BSAF, ICI, etc.), production using known methods, and the like.

The copolymer of 2-methacryloyloxyethylphosphocholine may be any copolymer comprising 2-methacryloyloxyethylphosphocholine and one or more other monomer components as constituent units, and is not particularly limited.

Specific examples of the copolymer of 2-methacryloyloxyethylphosphocholine include Lipidure®, Biolipidure®, equivalents of these products, and the like.

Examples of preferable copolymers of 2-methacryloyloxyethylphosphocholine include, but are not limited to, Lipidure BL-405, Lipidure BL-203, Lipidure BL-1002, Lipidure BL-103, Lipidure BL-206, Lipidure BL-802, equivalents of these products, and the like. Among these, Lipidure BL-206, Lipidure BL-802, equivalents of these products, and the like are most preferable (for example, Lipidure BL-802 is herein briefly referred to as Lipidure 802).

The copolymer of 2-methacryloyloxyethylphosphocholine contained in the blocking agent of the present invention may be a combination of two or more of the above compounds. Specific examples for obtaining such blocking agents include purchasing from the market (NOF Corporation), production using known methods, and the like.

The amount of hydroxyalkyl cellulose, polyvinyl alcohol, ethylene oxide-propylene oxide copolymer, or copolymer of 2-methacryloyloxyethylphosphocholine in the blocking agent of the present invention is not particularly limited. The total weight of the hydroxyalkyl cellulose, polyvinyl alcohol, ethylene oxide-propylene oxide copolymer, and copolymer of 2-methacryloyloxyethylphosphocholine may be about 0.001 to 100 parts by weight, based on 100 parts by weight of the blocking agent. Specifically, hydroxyalkyl cellulose, polyvinyl alcohol, an ethylene oxide-propylene oxide copolymer, or a copolymer of 2-methacryloyloxyethylphosphocholine itself may be used as the blocking agent of the present invention.

As long as the above effects are not impaired, the blocking agent according to the present invention may contain components that are other than the above hydroxyalkyl cellulose, polyvinyl alcohol, ethylene oxide-propylene oxide copolymer, and copolymer of 2-methacryloyloxyethylphosphocholine and that are used in an immunoassay. Specific examples of such other components include, but are not limited to, preservatives, pH adjusting agents, salts, surfactants, stabilizers, and the like. The blocking agent according to the present invention may be provided in the form of being contained in water, a buffer, or the like commonly used in immunoassays, or provided in the form of a solid that is used by being dissolved in water, a buffer, or the like before use.

The amount of blocking agent of the present invention to be used is not particularly limited. For example, the blocking agent may typically be used in an amount of about 0.0001 to 5 parts by volume, per 100 parts by volume of the solution used in an immunoassay, such as a buffer.

### Immunoassay Kit

The immunoassay kit according to the present invention comprises the virus-like particle of the present invention and the blocking agent of the present invention.

The virus-like particle and the blocking agent are as described in detail in the above Blocking Agent section. The immunoassay can be the same as the immunoassay described in detail in the above Virus-like Particle section.

The immunoassay kit according to the present invention may comprise a reaction container, a chromogenic or luminescent substrate, a reaction solution, a standard substance, a disposable instrument, manufacturer's instructions, and the like.

In the immunoassay kit according to the present invention, the virus-like particle of the present invention and the blocking agent of the present invention may be contained in individual containers (packs, bottles, etc.) or in the same container. The virus-like particle and the blocking agent may be provided in the form of being contained in water, a buffer, or the like, and in particular, the blocking agent may be provided in the form of a powder that is used by being prepared (diluted) with water, a buffer, etc., before use in an immunoassay.

### EXAMPLES

The following examples serve to further illustrate the present invention but should in no way be construed as limiting the scope of the invention.

### Production Example 1

### Preparation of BNC-L

BNC-L is a virus-like particle containing a self-assembling HBsAg protein comprising the amino acid sequence of SEQ ID NO: 1. BNC-L was produced by using the method disclosed in Japanese Patent No. 4,085,231 or Japanese Patent No. 4,936,272.

Specifically, a yeast that expresses BNC-L obtained in U.S. Pat. No. 4,085,231 was cultured, and the cultured cells were disrupted using glass beads. The disrupted cell suspension was heat-treated at 70° C. for 20 minutes. After the heat-treatment, the resulting cell suspension was centrifuged. The obtained supernatant was collected, then purified using a cellulofine sulfate column and a gel filtration column, and concentrated to a protein concentration of 0.2 mg/mL or more to obtain BNC-L.

BNC-L, which contains a protein having self-organization ability comprising the amino acid sequence of SEQ ID NO: 1, forms a particle and is reported to contain about 110 molecules of the protein per particle (Yamada et al., Vaccine 19, 3154-3163, 2001).

### Preparation Example 2

### Preparation of BNC-ZZ

BNC-ZZ is a virus-like particle containing a protein having self-organization ability of SEQ ID NO: 2 and having two antibody-binding domains (Z domains) of protein A inserted in tandem into the N terminus of the HBsAg protein contained in the BNC-L obtained in Production Example 1. BNC-ZZ was produced by using the method disclosed in Japanese Patent No. 4,212,921 and Japanese Patent No. 4,936,272.

Specifically, a yeast that expresses the BNC-ZZ obtained in U.S. Pat. No. 4,212,921 was cultured, and the cultured cells were disrupted using glass beads. The disrupted cell suspension was heat-treated at 70° C. for 20 minutes. After the heat-treatment, the resulting cell suspension was centrifuged. The obtained supernatant was collected, then purified using a porcine IgG column and a gel filtration column, and concentrated to a protein concentration of 0.2 mg/mL or more to obtain BNC-ZZ.

Analogy from the above BNC-L suggests that BNC-ZZ contains about 110 molecules of the protein having self-organization ability comprising the amino acid sequence of SEQ ID NO: 2 per particle. When BNC-ZZ is mixed with an antibody, a complex that retains the ability of the antibody to bind to an antigen is formed based on the binding of the antibody-binding domain of the particle and the FC domain of the antibody. An enzyme-labeled BNC-ZZ also forms a similar complex. Hereinafter this complex may be referred to as a mixed complex.

### Production Example 3

### Preparation of HRP-Labeled BNC-ZZ

The BNC-ZZ obtained in Production Example 2 was labeled with HRP using a kit for labeling with HRP via SH (Peroxidase Labeling Kit-SH, produced by Dojindo Laboratories, Inc.) and a kit for labeling with HRP via NH₂ (Peroxidase Labeling Kit-NH₂, produced by Dojindo Laboratories, Inc.) according to the manufacturer's instructions, and then purified using a gel filtration column. Two kinds of HRP-labeled BNC-ZZ particles were thus obtained.

BNC-ZZ labeled with HRP via SH is confirmed to have about 110 HRP molecules per particle. That is, the results suggest that SH of at least one cysteine residue in the protein consisting of the amino acid sequence of SEQ NO.: 2 is crosslinked with about one HRP molecule.

The BNC-ZZ used to produce HRP-labeled BNC-ZZ was labeled with HRP under mild conditions that do not affect the particle shape. The particle size was actually measured by dynamic light scattering and the results show that the particle size of BNC-ZZ before labeling was 54 nm, whereas the particle size after labeling was 58 nm. That is, not much difference was observed in particle size. It is thus clear that both of the NH₂ and SH groups labeled with HRP are those derived from amino acid residues exposed to the particle surface.

Based on SEQ ID NO: 2 showing the amino acid sequence of the protein contained in BNC-ZZ, for example, when BNC-ZZ is labeled with HRP via NH₂, the NH₂ is suggested to be an NH₂ group at the N-terminus or an NH₂ group on a side chain of any of the lysine residues at positions 1, 43, 67, 70, 98, 112, 113, 121, 125, 128, 156, 170, 171, 179, 308, and 327.

When BNC-ZZ is labeled with HRP via SH, for example, the SH is suggested to be an SH group of any of the cysteine residues at position 293, 307, 310, 323, 324, 325, 333, and 335.

### Example 1

An experiment was carried out to measure the HRP activity of HRP-labeled BNC-ZZ prepared by using the two kinds of methods described in Production Example 3 above. Using a SAT-blue solution (produced by Dojindo Laboratories) as an HRP substrate, absorbance at 492 nm (Abs 492 nm) was measured. Using the protein content calculated from measurement of absorbance at 280 nm, the specific activity was calculated.

The results show that BNC-ZZ labeled with HRP via NH₂ (hereinafter sometimes referred to as NH₂-HRP-labeled BNC-ZZ) had a specific activity of 0.351 unit/μg (U/μg), whereas BNC-ZZ labeled with HRP via SH (hereinafter sometimes referred to as SH-HRP-labeled BNC-ZZ) had a specific activity of 0.844 U/μg. That is, NH₂-HRP-labeled BNC-ZZ exhibited only about 41% of the activity of the SH-HRP-labeled BNC-ZZ. The unit shows a specific increase in absorbance at 492 nm as measured using SAT-Blue as a substrate under the above conditions.

This result suggests that the number of NH₂ groups to which a protein molecule called HRP can access at the level of being capable of labeling is less than that of SH groups. Since labeling with a larger amount of HRP molecules can be done via SH groups, it became clear that labeling via SH groups has greater potential for the application of HRP-labeled BNC-ZZ as a high-sensitivity antibody detection probe.

### Production Example 4

### Preparation of ALP-Labeled BNC-ZZ

The BNC-ZZ obtained in Production Example 2 was labeled with ALP using a kit for labeling with ALP via SH (Alkaline Phosphatase Labeling Kit-SH, produced by Dojindo Laboratories, Inc.) and a kit for labeling ALP via NH₂ (Alkaline Phosphatase Labeling Kit-NH₂, produced by Dojindo Laboratories, Inc.) according to the manufacturer's instructions, and purified using a gel filtration column. Two kinds of ALP-labeled BNC-ZZ were thus obtained.

BNC-ZZ used to produce ALP-labeled BNC-ZZ was labeled with ALP under such conditions as not to destroy the particle shape as in the production of the HRP-labeled BNC-ZZ in Production Example 3.

In the NH₂-ALP-labeled BNC-ZZ and SH-ALP-labeled BNC-ZZ, BNC-ZZ is thus suggested to have been labeled with ALP via NH₂ or SH of the same amino acid residue as in the above-mentioned HRP-labeled BNC-ZZ.

### Example 2

An experiment was carried out to measure the ALP activity of ALP-labeled BNC-ZZ produced by using the two kinds of methods described above in Production Example 4. Using pNPP (Sigma Fast p-nitro phenyl phosphate tablets) as an ALP substrate, absorbance at 405 nm (Abs 405 nm) was measured. Using the protein content calculated from measurement of absorbance at 280 nm, the specific activity was calculated.

The results show that the BNC-ZZ labeled with ALP via NH₂ (hereinafter sometimes referred to as “NH₂-ALP-labeled BNC-ZZ”) had a specific activity of ALP enzyme of 3.56 U/μg, whereas BNC-ZZ labeled with ALP via SH (hereinafter sometimes referred to as SH-ALP-labeled BNC-ZZ) had a specific activity of ALP enzyme of 5.61 U/μg. That is, NH₂-ALP-labeled BNC-ZZ exhibited only about 60% of the activity of SH-ALP-labeled BNC-ZZ. The unit shows a specific increase in absorbance at 405 nm when pNPP, which is a substrate, is reacted under the above conditions.

This result shows that labeling with a larger number of ALP molecules can be done via SH. It became clear that this labeling method has greater potential for the application of ALP-labeled BNC-ZZ as a high-sensitivity antibody detection probe.

### Production Example 5

### Preparation of AGG-BNC-ZZ

A protein comprising the amino acid sequence of SEQ ID NO: 6 consisting of one binding domain derived from protein A having the above-mentioned Z domains and two binding domains derived from protein G was prepared in E. coli (hereinafter this protein may be referred to as AGG). EMCS (produced by Dojindo Laboratories, Inc.) was added to an AGG protein solution to introduce a maleimide group into an amino group of AGG.

BNC-ZZ obtained in Production Example 2 was subjected to a reduction treatment using TCEP (produced by Thermo Scientific), and the reduced BNC-ZZ and AGG having a maleimide group introduced thereinto were incubated to proceed a crosslinking reaction, thus obtaining AGG-BNC-ZZ. Specifically, AGG-BNC-ZZ is a virus-like particle containing a protein comprising the amino acid sequence of SEQ ID NO: 2 and having AGG bound via cysteine residues of the protein.

### Production Example 6

### Preparation of HRP-Labeled AGG-BNC-ZZ

AGG-BNC-ZZ obtained in Production Example 5 was labeled with HRP using Peroxidase Labeling Kit-NH₂ according to the manufacturer's instructions to obtain HRP-labeled AGG-BNC-ZZ.

### Example 3

An experiment was carried out to compare the HRP enzyme activity of HRP-labeled AGG-BNC-ZZ obtained in Production Example 6 and the enzyme activity of SH-HRP-labeled BNC-ZZ obtained in Production Example 3. A predetermined amount of HRP-labeled AGG-BNC-ZZ or SH-HRP-labeled BNC-ZZ was collected and a TMB solution (One-Step TMB Ultra, produced by Thermo Scientific), which is an HRP substrate, was added thereto. Absorbance at 450 nm was measured.

The results show that the HRP specific activity of the HRP-labeled AGG-BNC-ZZ is approximately one third of that of HRP-labeled BNC-ZZ. As is seen from the results of Example 2, in which BNC-ZZ was labeled with HRP via NH₂ or SH and HRP activity was investigated, it became clear that AGG-BNC-ZZ can also be labeled with HRP via NH₂ with nearly the same efficiency as AGG-BNC-ZZ and BNC-ZZ.

### Production Example 7

### Preparation of HRP-Labeled BNC-ZZ/Rabbit-Derived Anti-Mouse IgG Antibody Complex

As described in Production Example 2, when BNC-ZZ is mixed with an antibody, a mixed complex is formed. The binding of the Fc region of the antibody to the antibody-binding site of BNC-ZZ in the mixed complex is reversible; therefore, in the presence of plural kinds of antibodies, an antibody bound may be replaced with another. Accordingly, if this binding is made irreversible, the binding capacity of the antibody bound is expected to be imparted to BNC-ZZ. Accordingly, a product in which the binding of the antibody-binding domain and the antibody was crosslinked was prepared in the following manner. Specifically, SH-HRP-labeled BNC-ZZ obtained in Production Example 3 was mixed with a rabbit-derived anti-mouse IgG antibody (produced by Bethyl) to form a complex thereof. Further, BS₃ (produced by Dojindo Laboratories, Inc.) as a crosslinking agent was added to a final concentration of 0, 50, 200, 400, or 1,000 μM. Subsequently, an excess of the rabbit-derived anti-mouse IgG antibody was removed using a Protein A Sepharose resin (produced by GE Healthcare) to obtain an HRP-labeled BNC-ZZ/rabbit-derived anti-mouse IgG antibody complex.

### Example 4

An experiment was carried out to measure the HRP activity of the HRP-labeled BNC-ZZ/rabbit-derived anti-mouse IgG antibody complex obtained in Production Example 7. Using the HRP-labeled BNC-ZZ/antibody complex, HRP activity was measured by the method described in Example 3. Table 1 shows the results. The absorbance at 450 nm was similar at all of the concentrations of the crosslinking agent. The results show that HRP activity hardly changes.

TABLE 1

| | Absorbance at 450 nm |
|---|---|
| Various Samples | (relative value) |
| Crosslinking agent 0 μM | 1.000 |
| (no crosslinking agent) | |
| Crosslinking agent 50 μM | 0.935 |
| Crosslinking agent 200 μM | 0.966 |
| Crosslinking agent 400 μM | 0.998 |
| Crosslinking agent 1,000 μM | 0.978 |

### Example 5

A competition experiment was carried out to evaluate the binding behavior of an antibody to an SH-HRP-labeled BNC-ZZ/antibody complex. IgG with no antigen binding properties, which was obtained from normal mouse serum by purification using Protein A/G Sepharose (produced by GE Healthcare) (hereinafter sometimes referred to as “control mouse-derived IgG”) was added at various concentrations to wells of an ELISA plate and immobilized. The plate was then blocked using k-Block-e (produced by Beacle, Inc.)

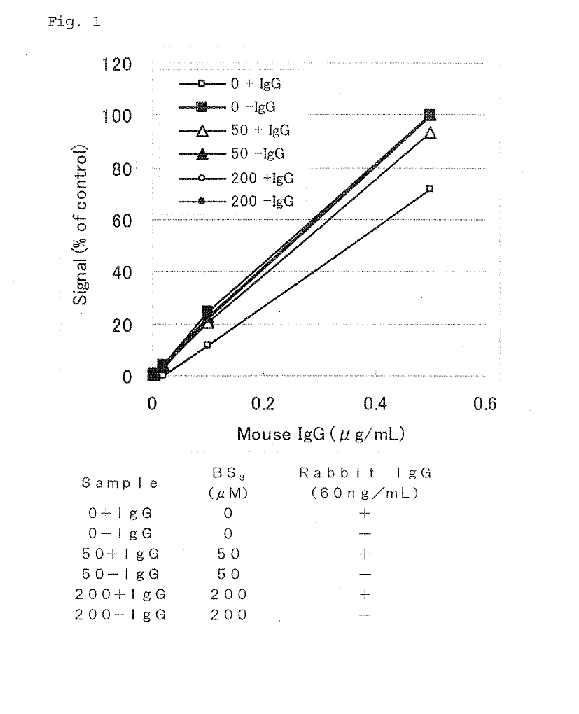

The HRP-labeled BNC-ZZ/rabbit-derived anti-mouse IgG antibody complex obtained in Production Example 7 and the control rabbit-derived IgG (produced in the same manner as above by our company) were mixed and incubated. The resulting mixture was then added to the immobilized wells and a reaction was allowed to proceed. After washing, absorbance at 450 nm was measured in the same manner as in Example 3. FIG. 1 shows the results.

When an HRP-labeled BNC-ZZ/anti-mouse IgG rabbit-derived antibody complex was prepared using a 0M crosslinking agent, i.e., without being subjected to crosslinking, the reaction with immobilized mouse-derived IgG in the presence of control rabbit-derived IgG was reduced to 70% of the reaction in the absence of the control rabbit-derived IgG over the entire concentration range. When an HRP-labeled BNC-ZZ/rabbit-derived anti-mouse IgG antibody complex was crosslinked using 50 μM BS₃, the reaction with immobilized mouse-derived IgG was slightly reduced in the presence of the control rabbit-derived IgG, which indicates that some reversible bonds remained. When the control rabbit-derived IgG was added to a complex crosslinked with 200 μM BS₃, no change was observed in reaction with the mouse-derived IgG. This indicates that in the HRP-labeled BNC-ZZ/rabbit-derived anti-mouse IgG antibody complex crosslinked with 200 μM BS₃, no replacement of the antibody bound to the complex occurred and that a complex was formed by irreversible bonding.

The above results clearly show that when an HRP-labeled BNC-ZZ/rabbit-derived anti-mouse IgG antibody complex is crosslinked with BS₃ at a concentration of 200 μM or higher, a complex can be formed by irreversible bonding.

### Example 6

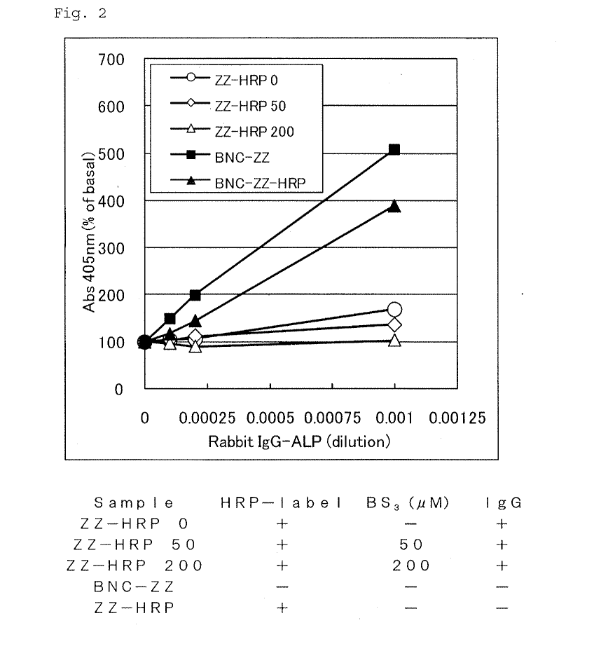

An experiment was carried out to evaluate the residual antibody binding activity and reversible antibody binding activity of the HRP-labeled BNC-ZZ/rabbit-derived anti-mouse IgG antibody complex obtained in Production Example 7. A complex was produced in the same manner as the method of producing an HRP-labeled BNC-ZZ/rabbit-derived anti-mouse IgG antibody complex treated with a crosslinking agent at various concentrations described in Production Example 7 except that a complex with a control rabbit-derived IgG was produced in place of a complex with a rabbit-derived anti-mouse IgG antibody. BS₃ was used at concentrations of 0 μM, 50 μM, and 200 μM. These complexes were added to wells of an ELISA plate and immobilized. The plate was blocked with k-Block-e (produced by Beacle, Inc.). Subsequently, an ALP-labeled rabbit-derived anti-mouse IgG antibody (produced by Invitrogen) diluted to 1:10,000, 1:5,000, and 1:1,000 was added to the wells, and a reaction was allowed to proceed. After washing, absorbance at 405 nm was measured in the same manner as in Example 2. FIG. 2 shows the results.

BNC-ZZ and HRP-labeled BNC-ZZ exhibited an increased ALP enzyme activity depending on the amount of ALP-labeled rabbit antibody added. When a 1:1,000 dilution of the ALP-labeled rabbit antibody was added, the ALP enzyme activity increased four times or more, compared to no addition of the antibody. This indicates that the ALP-labeled rabbit antibody was bound to the antibody binding sites of BNC-ZZ and HRP-labeled BNC-ZZ. In contrast, when the 1:1,000 dilution of the ALP-labeled rabbit antibody was added to a mixed complex (“ZZ-HRP 0” in FIG. 2) formed by merely mixing HRP-labeled BNC-ZZ and a control rabbit-derived IgG beforehand or to a complex crosslinked by adding 50 μM BS₃ (“ZZ-HRP 50” in FIG. 2), the former complex exhibited a 170% ALP activity, and the latter complex exhibited a 137% ALP activity, compared to no addition. The results show that the ALP-labeled rabbit antibody can bind to these complexes. In contrast, when the complex is crosslinked with 200 μM BS₃ (“ZZ-HRP 200” in FIG. 2), no binding of the ALP-labeled rabbit antibody was observed.

The above results show that when a complex of HRP-labeled BNC-ZZ and antibody is crosslinked with BS₃ at a concentration of 200 μM or more, the HRP-labeled BNC-ZZ has no remaining antibody binding activity and contains no reversible bonds.

### Production Example 8

### Preparation of HRP-Labeled BNC-SA/Anti-Mouse IgG Antibody Complex

A maleimide group (MAL group) was introduced into the amino group of streptavidin (SA, produced by Thermo Scientific Inc.) using EMCS. Subsequently, BNC-L obtained in Production Example 1 and SA having the MAL group introduced thereinto were incubated to allow a crosslinking reaction to proceed, thus obtaining an SA-labeled BNC-L (hereinafter sometimes referred to as BNC-SA). Specifically, BNC-SA is a virus-like particle containing a protein consisting of the amino acid sequence of SEQ: NO:1 and having SA bound via at least one cysteine residue of the protein. Further, the obtained BNC-SA was crosslinked with HRP using a Peroxidase Labeling Kit-NH₂ to obtain HRP-labeled BNC-SA. Further, using a Biotin Labeling Kit-NH₂ (produced by Dojindo Laboratories, Inc.), a rabbit-derived biotinylated anti-mouse IgG antibody was obtained. The HRP-labeled BNC-SA and the rabbit-derived biotinylated anti-mouse IgG antibody were mixed in an equal amount in terms of the amount of protein to obtain an HRP-labeled BNC-SA/rabbit-derived anti-mouse IgG antibody complex.

### Example 7

An experiment was carried out to measure the HRP activity of an HRP-labeled BNC-SA/anti-mouse IgG antibody complex. The HRP activity of the HRP-labeled BNC-SA/rabbit-derived anti-mouse IgG antibody complex obtained in Production Example 8 was measured in the same manner as in Example 1 above.

As a result, it was found that the HRP activity of the HRP-labeled BNC-SA/rabbit-derived anti-mouse IgG antibody complex is about 1/8 of that of the control SH-HRP-labeled BNC-ZZ. Since the HRP activity of NH₂-HRP-labeled BNC-ZZ is about 1/2.4 of that of SH as is seen from the results of Example 1, the HRP activity of the HRP-labeled BNC-SA/rabbit-derived anti-mouse IgG antibody can be determined to be about 1/3 of that of NH₂-HRP-labeled BNC-ZZ.

### Example 8

An experiment was carried out to investigate the adsorption of BNC-ZZ on a container and the effects of blocking agents. A solution prepared by dissolving BNC-ZZ obtained in Production Example 2 in PBS to a concentration of 300 ng/mL was pipetted into microtubes made of polyethylene, and various blocking agents shown in Table 2 were added. After each tube was sealed, the tube was left at room temperature for 4 days while being rotated. The amount of BNC-ZZ remaining in the solution was then measured. The measurement was performed using an ELISA kit for measuring the Pre-S1 region on the particle surface of the BNC-ZZ (HB Pre-S1 Antigen Quantitative ELISA Kit, Rapid, produced by Beacle, Inc.) according to the manufacturer's instructions. Table 2 shows the results.

When only PBS was used, 1.3% of BNC-ZZ remained in the solution. Most of the BNC-ZZ was thus found to be adsorbed on the container. When various blocking agents were added thereto, all of the blocking agents exhibited inhibitory effects. Among these, skim milk, Blockace (produced by Dainippon Pharmaceutical Co., Ltd.), Pluronic F-127, and Lipidure 802 exhibited high inhibitory effects.

TABLE 2

| | Percentage of BNC-ZZ remaining |
|---|---|
| Various blocking agents | in the solution (%) |
| PBS (no blocking agent) | 1.3 |
| 0.5% skim milk | 92.3 |
| 0.4% Blockace | 92.5 |
| 0.1% Pluronic F-127 | 89.3 |
| 0.1% Pluronic P-105 | 77.0 |
| 0.1% HPMC | 52.8 |
| 0.1% PVA-2000 | 57.2 |
| 0.1% Lipidure 206 | 61.6 |
| 0.1% Lipidure 802 | 81.9 |

### Example 9

An experiment was carried out to investigate the adsorption of HRP-labeled BNC-ZZ on a container and the effects of blocking agents. PBS solutions containing the SH-HRP-labeled BNC-ZZ of Production Example 3 at a final concentration of 300 ng/mL with various blocking agents shown in Table 3 were prepared and pipetted into polyethylene tubes without surface treatment, tubes treated with MPC (2-methacryloyloxyethyl phosphorylcholine), and glass tubes. All of the tubes were sealed and left at 4° C. for 2 days while being rotated. The HRP activity of each solution was measured in the following manner. Specifically, 2 μg/mL of pig IgG was added to wells of an ELISA 96-well microplate and immobilized. Subsequently, the plate was blocked by adding 1% Blockace. The HRP-labeled BNC-ZZ solutions left in the above-mentioned various tubes were added to the wells and bound to the immobilized pig IgG. Subsequently, after a TMB solution (1-Step TMB slow: produced by Thermo Scientific) was added, absorbance at 450 nm was measured using a plate reader. The percentage of HRP-labeled BNC-ZZ remaining in each sample was calculated based on the absorbance at 450 nm of a dilution obtained by diluting 500 μg/mL of a control sample similarly stored in an MPC-treated tube to 300 nm/mL. Table 3 shows the results.

TABLE 3

| | Treatment | | | | | |
|---|---|---|---|---|---|---|
| | | | | | 0.1% | 0.1% |
| | | 0.1% | 0.1% | 0.1% | Pluronic | Pluronic |
| tube | PBS | gelatin | BSA | HPMC | F127 | P105 |
| non-treated | 22.3 | 66.3 | 88.5 | 84.2 | 92.3 | 93.3 |
| PE tube | | | | | | |
| MPC-treated | 89.1 | 95.5 | 99 | 95.5 | 99.5 | 97.8 |
| PE tube | | | | | | |
| glass tube | 52.4 | 77.6 | 94.3 | 87.6 | 97.5 | 90.3 |

When the solution containing no blocking agent (PBS in Table 3) was placed in the non-treated plastic tube, 78% was adsorbed on the tube in 2 days. When the solution was placed in the glass tube, 48% was adsorbed on the tube. Although the adsorption on the MPC-treated tube was suppressed, 10% was still adsorbed. In contrast, when various blocking components were added, inhibitory effects were observed in each of the additions. In particular, the addition of BSA, HPMC, Pluronic F-127, or Pluronic P-105 achieved high inhibitory results.

### Example 10

An experiment was carried out to investigate the adsorption of BNC-ZZ on a PVDF membrane and the effects of blocking agents. A PVDF membrane was cut into small pieces and subjected to blocking treatment by immersion into PBS containing a blocking agent at various concentrations shown in Table 4 at room temperature for 1 hour, and the blocking agent not bound to the membrane was removed by washing with PBS-T three times. Subsequently, a reaction solution prepared by dissolving the BNC-ZZ obtained in Production Example 2 to a concentration of 300 ng/mL in PBS-T containing a blocking agent at various concentrations as shown in Table 4 was allowed to react with the blocked PVDF film at room temperature for 1 hour and then reacted with an HRP-labeled rabbit antibody dissolved in PBS-T at room temperature for 20 minutes.

After the reaction was completed, the resulting product was washed with PBS-T five times and reacted with ECL Prime (produced by GE Healthcare), which is an HRP luminescent substrate. Luminescence was measured using a luminescence sensor (ChemiDoc XRS, produced by Bio-Rad) by exposure to light for 20 minutes.

As a control, BNC-ZZ treated in the same manner as above using PBS-T containing no blocking component was used. Table 4 shows the results.

TABLE 4

| Blocking agents used | | Results |
|---|---|---|
| PVDF membrane | Reaction solution | Signal intensity |
| PBS | PBS-T | +++++ |
| 5% Skim milk | 1% Skim milk | − |
| 4% Blockace | 1% Blockace | − |
| 5% BSA | 1% BSA | − |
| 1% Pluronic F-127 | 0.1% Pluronic F-127 | − |
| 1% Pluronic P-105 | 0.1% Pluronic P-105 | − |
| 1% HPMC | 0.1% HPMC | − |
| 1% PVA2000 | 0.1 PVA 2000 | − |
| 1% PVA500 | 0.1% PVA 500 | − |
| 1% Lipidure 206 | 0.1% Lipidure 206 | − |
| 1% Lipidure 802 | 0.1% Lipidure 802 | − |

* The signal intensity was evaluated from the intensity of the obtained image using a luminescence sensor and rated on a six-stage scale of −, +, ++, +++, ++++, and ++++++. “—” indicates that no signal was obtained.

The entire image of the PVDF membrane treated with PBS containing no blocking agent became dark, which clearly shows that a large amount of BNC-ZZ was adsorbed on the PVDF membrane.

In contrast, when a PVDF membrane was subjected to blocking treatment using various blocking agents and the same blocking agent was also incorporated into the BNC-ZZ solution to be reacted, nearly no adsorption of BNC-ZZ on the PVDF membrane was observed regardless of the type of blocking agent used.

The above results clearly show that all of the blocking agents used in this experiment exhibit blocking effects when used for both of the blocking treatments, i.e., one by treating the PVDF membrane and another by addition to the reaction solution.

### Example 11

An experiment was carried out to investigate the adsorption of HRP-labeled BNC-ZZ on a PVDF membrane and the effects of blocking agents. Solutions (membrane blocking solutions) of 5% skim milk, 10% skim milk+3% fish gelatin (produced by Funakoshi), 4% Blockace, and 5% bovine serum albumin in TBS were prepared. A PVDF membrane was cut into small pieces and subjected to blocking treatment using the membrane blocking solutions. An untreated PVDF membrane was used as a control for the membrane blocking treatment.

Subsequently, TBS-T containing the SH-HRP-labeled BNC-ZZ obtained in Production Example 3 was prepared, and 1% skim milk, 1% Blockace, 1% BSA, or 2% gelatin was added to prepare a reaction blocking solution. The reaction blocking solution was added to the PVDF membrane. TBS-T containing SH-HRP-labeled BNC-ZZ was used as a control. After the addition, the PVDF membrane was appropriately washed and subjected to detection in the same manner as in Example 10. Table 5 shows the results.

TABLE 5

| | | Reaction blocking solution | | | | |
|---|---|---|---|---|---|---|
| | | | 1% | 1% | 1% | 1% |
| | | Control | Skim milk | Blockace | BSA | Gelatin |
| Membrane | Control | +++++ | + | + | +++ | + |
| blocking | 5% Skim | +++ | + | + | +++ | + |
| solution | milk | | | | | |
| | 4% Skim | +++ | + | + | +++ | + |
| | milk | | | | | |
| | 5% BSA | +++ | + | + | ++++ | + |
| | 10% Skim | +++ | + | + | +++ | ++ |
| | milk + 3% | | | | | |
| | gelatin | | | | | |

* The signal intensity was evaluated from the intensity of the obtained image using a luminescence sensor and rated on a six-stage scale of −, +, ++, +++, ++++, and ++++++.
“−” indicates that no signal was obtained.

When the PVDF membrane was not subjected to blocking treatment and TBS-T containing HRP-labeled BNC-ZZ was added, the entire image became dark, and the score of its signal intensity was +++++. This indicates that a large amount of the HRP-labeled BNC-ZZ was adsorbed on the PVDF membrane. In contrast, when TBS-T containing HRP-labeled BNC-ZZ was added to the PVDF membrane treated with various membrane blocking solutions, adsorption of HRP-labeled BNC-ZZ on the PVDF membrane was suppressed, but a clear adsorption reaction was still observed. When the PVDF membrane was treated with a membrane blocking solution and then the same blocking solution was further added, the adsorption of HRP-labeled BNC-ZZ on the PVDF membrane was strongly inhibited, compared to the control, in all of the cases except for the reaction blocking solution containing BSA. When the reaction blocking solution containing BSA was used, the PVDF membrane exhibited a high level of signal intensity regardless of the type of blocking treatment to which the PVDF membrane was subjected, which is different from the results obtained using the other reaction blocking solutions.

The above results show that the blocking treatment to the PVDF membrane is important for inhibiting the adsorption of HRP-labeled BNC-ZZ on the PVDF membrane, and the type of reaction blocking solution used is even more important. The results further show that all of the blocking solutions exhibit similar levels of effects when used for the treatment of the PVDF membrane; however, when used as reaction blocking solutions, Blockace, skim milk, and fish gelatin exhibit almost similar levels of effects, whereas BSA has a weak effect.

### Example 12

An experiment was carried out to investigate the adsorption of HRP-labeled BNC-ZZ on the PVDF membrane and the effects of chemical blocking agents. Various blocking agents shown in Table 6 were dissolved in TBS to a concentration of 1%. As a control, a solution of 5% skim milk in TBS was prepared. Using these, the PVDF membrane was blocked. Subsequently, the blocked PVDF membrane was reacted with SH-HRP-labeled BNC-ZZ obtained in Production Example 3 and evaluated in the same manner as in Example 11. Table 6 shows the results.

The entire image of the PVDF membrane blocked with 5% skim milk was slightly dark, and adsorption on the HRP-labeled BNC-ZZ membrane was not completely inhibited. When the membrane was blocked with Pluronic F-127, Pluronic P-105, HPMC, PVA2000, PVA500, Lipidure206, or Lipidure802 among the investigated compounds, nearly no darkening of the membrane occurred and strong inhibitory effects were observed.

TABLE 6

| Various blocking agents | Signal intensity |
|---|---|
| 5% Skim milk | ++ |
| 1% Pluronic F-127 | − |
| 1% Pluronic P-105 | − |
| 1% CMC | ++ |
| 1% HPMC | − |
| 1% PVP K-25 | +++ |
| 1% PVP K-70 | ++ |
| 1% PVA2000 | − |
| 1% PVA500 | − |
| 1% Lipidure 103 | ++ |
| 1% Lipidure 206 | − |
| 1% Lipidure 802 | − |

* The signal intensity was evaluated from the intensity of the obtained image using a luminescence sensor and rated on a six-stage scale of −, +, ++, +++, ++++, and ++++++. “—” indicates that no signal was obtained.

### Example 13

An experiment was carried out to investigate the effect of HRP-labeled BNC-ZZ in Western blotting. The results of Examples 11 and 12 suggest that when SH-HRP-labeled BNC-ZZ is used with a PVDF membrane, using Pluronic F-127, Pluronic P-105, HPMC, PVA2000, PVA500, Lipidure206, or Lipidure802 as a blocking agent can inhibit the adsorption of HRP-labeled BNC-ZZ on the membrane. The results of Example 10 suggested that when the reaction solution of HRP-labeled BNC-ZZ contains a blocking agent, a higher effect can be achieved.

Accordingly, the effect of incorporating these blocking agents into the reaction solution was investigated by subjecting an HuH7 cell extract to Western blotting using an anti-Vimentin antibody and SH-HRP-labeled BNC-ZZ obtained in Production Example 3. The detection method was the same as in Example 10. The membrane was blocked using 5% skim milk. Various blocking agents shown in Table 7 below were used at a final concentration of 1% for blocking in the reaction.

TABLE 7

| Reaction blocking | | |
|---|---|---|
| agent | Band | Background |
| Skim milk | ++ | ++ |
| Pluronic F-127 | ++ | − |
| Pluronic P-105 | ++ | + |
| HPMC | ++ | + |
| PVA 2000 | ++ | − |
| PVA 500 | ++ | − |
| Lipidure 206 | ++ | + |
| Llpidure 802 | ++ | − |

* The signal intensity was evaluated from the intensity of the obtained image using a luminescence sensor and rated on a six-stage scale of −, +, ++, +++, ++++, and ++++++. “−” indicates that no signal was obtained.

In the control experiment in which 1% skim milk was added, the entire image of the membrane was slightly darkened as a background. In contrast, when Pluronic F-127, Pluronic P-105, HPMC, PVA2000, PVA500, Lipidure 206, or Lipidure 802 was added to the reaction solution, the background was lower than that of the control and the addition of each of the above blocking agents was found to be effective for inhibiting adsorption, although there are some differences in effect depending on the type of blocking agent used. On the other hand, the signal intensity of the antibody-specific band hardly changed, regardless of the type of blocking agent used.

The above results show that the addition of a blocking agent to the HRP-labeled BNC-ZZ reaction solution can inhibit non-specific adsorption of HRP-labeled BNC-ZZ, whereas the antigen-specific signal is maintained.

### Example 14

An experiment was carried out to investigate the effects of various blocking agents on the specific binding of probes in ELISA. Control mouse-derived IgG (polyclonal) was immobilized on an ELISA plate. As a control, an ELISA plate on which control mouse-derived IgG was not immobilized was prepared. Both of the plates were prepared by being blocked with k-Block-e.

Various probes (BNC-ZZ obtained in Production Example 2, SH-HRP-labeled BNC-ZZ obtained in Production Example 3, HRP-labeled AGG-BNC-ZZ obtained in Production Example 6, HRP-labeled BNC-ZZ/rabbit-derived anti-mouse IgG antibody complex obtained in Production Example 7, and HRP-labeled BNC-SA/rabbit-derived anti-mouse IgG antibody complex obtained in Production Example 8) were added to these plates to a specific concentration, and their binding reaction with immobilized control mouse derived IgG was observed.

Various blocking agents shown in Table 8 were used at a final concentration of 0.1% with various probes. As a control for blocking agents, PBS-T containing casein at a final concentration of 0.2% was used.

BNC-ZZ and the HRP-labeled rabbit-derived control IgG antibody were simultaneously added and allowed to react and then washed. Absorbance at 450 nm was measured in the same manner as in Example 9. The measurement value was shown per probe as a specific reaction value calculated by defining the reaction of a reaction solution containing an immobilized antibody but not containing a blocking component as 100% and subtracting a measurement value obtained without using the immobilized antibody as a blank value. Table 8 shows the results.

TABLE 8

| | | Pluronic | Pluronic | | PVA | Lipidure | Lipidure | |
|---|---|---|---|---|---|---|---|---|
| | none | F127 | P105 | HPMC | 500 | 206 | 802 | Casein |
| BNC-ZZ + IgG-HRP | −3.6 | 23.8 | 29.1 | 6.2 | 0.9 | 32.5 | 22.3 | 14.9 |
| HRP-labeled BNC-ZZ | −1.5 | 17.2 | 46.4 | 23.3 | −12.1 | 67.3 | 45.5 | 51.5 |
| HRP-labeled AGG-BNC-ZZ | 0.8 | 36.0 | 60.1 | 3.0 | −1.4 | 66.1 | 61.6 | 17.3 |
| HRP-labeled BNC-ZZ IgG | −4.5 | 40.8 | 66.1 | 6.9 | 0.7 | 64.8 | 50.4 | 26.9 |
| conjugate BS3 50 μM | | | | | | | | |
| HRP-labeled BNC-ZZ IgG | 0.0 | 27.3 | 48.7 | 8.9 | 0.0 | 52.9 | 31.1 | 13.5 |
| conjugate BS3 200 μM | | | | | | | | |
| HRP-labeled BNC-ZZ IgG | −10.4 | 16.8 | 41.3 | 4.5 | 0.4 | 46.9 | 24.2 | 9.3 |
| conjugate BS3 400 μM | | | | | | | | |
| HRP-labeled BNC-SA-IgG | 2.2 | 4.9 | 10.9 | 0.6 | −1.6 | 13.6 | 6.2 | 3.0 |
| conjugate | | | | | | | | |

When the antibody and each of the probes were reacted without adding any blocking component, nonspecific binding was very high and specific reactions were hardly observed. SH-HRP-BNC-ZZ exhibited a considerable level of specific reaction with casein used as a control, but the reaction with other probes was low. When HPMC or PVA was used as a blocking agent, a low specific reaction was obtained. When other blocking agents were used, there were cases in which good specific reactions were observed although the magnitude of the reaction is different depending on the probe used.

### Example 15

Next, an experiment was carried out to investigate the adsorption of various probes on an ELISA plate and the effects of various blocking agents on specific reactions by using the blocking agent of Example 14 at various concentrations. The GFP protein prepared using E. coli was immobilized on an ELISA plate and blocked using k-Block-e.

Various probes (e.g., a mixed complex of rabbit-derived anti-GFP antibody and SH-HRP-labeled BNC-ZZ obtained in Production Example 3; a mixed complex of rabbit-derived anti-GFP antibody and HRP-labeled AGG-BNC-ZZ obtained in Production Example 6; and an HRP-labeled BNC-ZZ/rabbit-derived anti-GFP antibody complex) to which a variety of blocking agents had been added were added to the plate. Their binding reaction with the antigen was observed. Various blocking agents shown in Table 9 were used at final concentrations of 0.01%, 0.05%, and 0.1% to conduct investigations under three conditions.

The HRP-labeled BNC-ZZ/rabbit-derived anti-GFP antibody complex was prepared in the same manner as in Production Example 7 except that BS₃ was used at a concentration of 1,000 μM and a rabbit-derived anti-GFP antibody was used in place of the rabbit-derived anti-mouse IgG antibody. A control was prepared using the above control rabbit-derived IgG in place of the anti-GFP rabbit antibody. Absorbance at 450 nm was measured in the same manner as in Example 9.

Table 9 shows the results. The value obtained in the absence of an anti-GFP antibody was defined as a noise value. The value obtained in the presence of anti-GFP antibody was defined as a specific signal value. The numerical value obtained by dividing each signal value by each noise value is shown as an S/N ratio in Table 9.

TABLE 9

| | | Signal/noise ratio | | |
|---|---|---|---|---|
| | | | | HRP-labeled |
| | | HRP-labeled | HRP-labeled | BNC-ZZ/ |
| | | BNC-ZZ | AGG-BNC-ZZ | IgG 1000 |
| Pluronic F127 | 0.10% | 6.5 | 4.2 | 5.4 |
| | 0.05% | 10.6 | 5.8 | 8.2 |
| | 0.01% | 9.6 | 7.2 | 8.9 |
| Pluronic P105 | 0.10% | 8.2 | 9.6 | 5.6 |
| | 0.05% | 8.4 | 7.2 | 6.4 |
| | 0.01% | 8.5 | 12.4 | 10.8 |
| HPMC | 0.10% | 6.5 | 6.1 | 8.3 |
| | 0.05% | 6.4 | 6.3 | 12.7 |
| | 0.01% | 8.5 | 8.1 | 14.3 |
| PVA-500 | 0.10% | 5.9 | 6.3 | 10.4 |
| | 0.05% | 5.6 | 7.1 | 9.3 |
| | 0.01% | 9.9 | 10.1 | 13.3 |
| Lipidure 206 | 0.10% | 12.3 | 6.9 | 6.5 |
| | 0.05% | 13.1 | 8.6 | 12.0 |
| | 0.01% | 9.6 | 9.2 | 11.7 |
| Lipidure 802 | 0.10% | 12.2 | 7.7 | 13.5 |
| | 0.05% | 9.3 | 6.9 | 13.5 |
| | 0.01% | 9.7 | 7.0 | 9.3 |

Various blocking agents provided different S/N ratios for each probe. Some exhibited high S/N ratios when the concentrations of blocking agents were high. Conversely, others exhibited high S/N ratios when the concentrations of the blocking agents were low. The results suggest that all of these blocking agents, when used at appropriate concentrations, are effective for using BNC-ZZ or the like in ELISA.

### Example 16

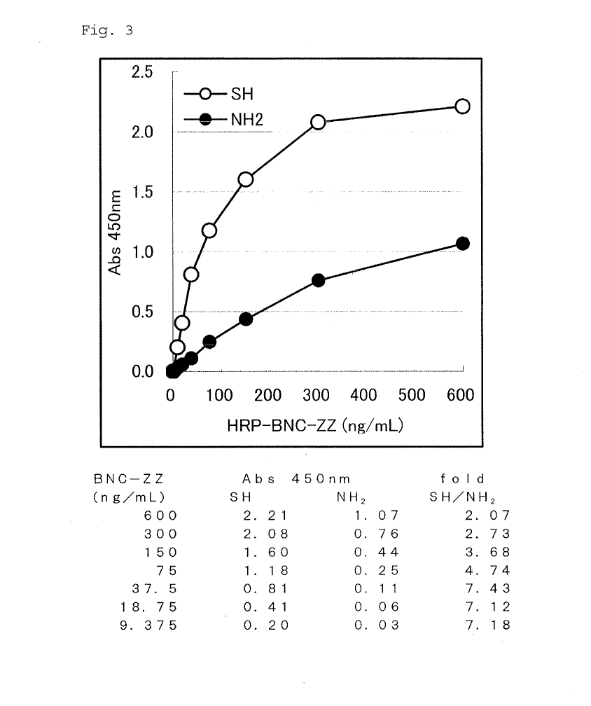

An experiment was carried out to evaluate the antibody binding activity of HRP-labeled BNC-ZZ. The NH₂-HRP-labeled BNC-ZZ and SH-HRP-labeled BNC-ZZ prepared in Production Example 3 were compared for antibody binding activity in ELISA. The control rabbit-derived IgG was immobilized on an ELISA plate and the wells of the plate were blocked with 1% Blockace. Solutions of HRP-labeled BNC-ZZ at concentrations of 0, 9.375, 18.75, 37.5, 75, 150, 300, and 600 ng/ml, in terms of the amount of BNC-ZZ protein were prepared. PBS-T containing Pluronic F-127 at a final concentration of 0.05% was added to the wells and a reaction was allowed to proceed. After washing, absorbance at 450 nm was measured in the same manner as in Example 9. FIG. 3 shows the results.

At all of the concentrations, the measurement values obtained using SH-HRP-labeled BNC-ZZ were higher than those obtained using NH₂-HRP-labeled BNC-ZZ. In particular, at the concentrations of 300 ng/mL and 600 ng/mL, the measurement values obtained using SH-HRP-labeled BNC-ZZ were about 2.1 to 2.7 times higher than those obtained using NH₂-HRP-labeled BNC-ZZ. This multiplication factor is almost the same as the level of difference in HRP specific activity therebetween shown in Example 1 above (since the former has an HRP specific activity of 0.844 U/μg and the latter has an HRP specific activity of 0.351 U/μg, the former is about 2.4 times higher than the latter). The results suggest that both of SH-HRP-labeled BNC-ZZ and NH₂-HRP-labeled BNC-ZZ were bound substantially in the maximum amount to the immobilized antibody at these concentrations. In contrast, when the concentration is lower than this level, the multiplication factor of the measurement value obtained using SH-HRP-labeled BNC-ZZ relative to the value obtained using NH₂-HRP-labeled. BNC-ZZ increased. When the concentration of HRP-labeled BNC-ZZ was 37.5 ng or less, the measurement value obtained using SH-HRP-labeled BNC-ZZ was about 7.24 times higher on the average. This indicates that in low concentration regions, SH-HRP-labeled BNC-ZZ was bound to the immobilized antibody in a larger amount. Considering the fact that the difference in HRP specific activity is about 2.4 times, the results suggest that the binding affinity of SH-HRP-labeled BNC-ZZ for the antibody is about three times higher than that of NH₂-HRP-labeled BNC-ZZ. The labeling via NH₂ is mainly performed by targeting lysine residues of the BNC-ZZ protein forming particles. Since lysine residues are abundant at antibody binding sites, labeling via NH₂ groups is considered to affect the antibody binding sites and thus inhibit the binding of the antibody. In contrast, since SH groups are present at the externally exposed sites in the transmembrane domain of the BNC-ZZ protein, labeling by targeting SH groups is considered to not affect antibody binding capacity.

Accordingly, when BNC-ZZ is labeled with HRP, labeling via SH can make better use of the antibody binding capacity of BNC-ZZ. This also provides high HRP activity, and can form a marker substance that is about 7.2 times higher in terms of the binding activity to the antibody and the HRP labeling. Accordingly, labeling via SH can be concluded to be much superior. Hereinafter the HRP-labeled BNC-ZZ refers to labeling via SH groups, unless otherwise specified.

### Example 17

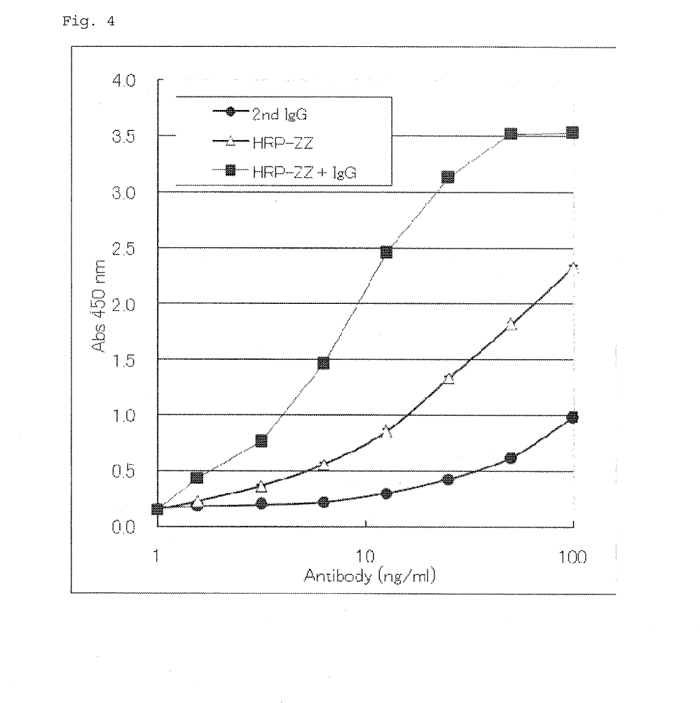

An experiment was carried out to investigate the application of HRP-labeled BNC-ZZ to ELISA. Pre-S2 (product number BCL-AGS 2-21, produced by Beacle, Inc.), which is a peptide of the surface antigen of a hepatitis B virus, was immobilized on an ELISA plate, and the plate was blocked with k-Block-e. An anti-Pre-S2 mouse antibody (2APS42, produced by the Institute of Immunology, Co., Ltd.) was added at various concentrations. Subsequently, an HRP-labeled anti-mouse antibody dissolved in PBS-T containing Pluronic F-127 at a final concentration of 0.05%, HRP-labeled BNC-ZZ of Production Example 3, or a combination of the HRP-labeled anti-mouse antibody and HRP-labeled BNC-ZZ at the same concentration was added, and a reaction was allowed to proceed. After washing, absorbance at 450 nm was measured by the same method as in Example 9. FIG. 4 shows the results.

The results show that the use of the HRP-labeled BNC-ZZ as indicated by “HRP-ZZ” with white triangles in FIG. 4 achieves an approximately 10-fold higher sensitivity, compared to detection with the anti-mouse antibody as indicated by “2nd IgG” with black circles in FIG. 4, and that the combined use of ZZ and IgG as indicated by “HRP-ZZ+IgG” with black squares in FIG. 4 achieves an approximately 30-fold higher sensitivity.

### Example 18

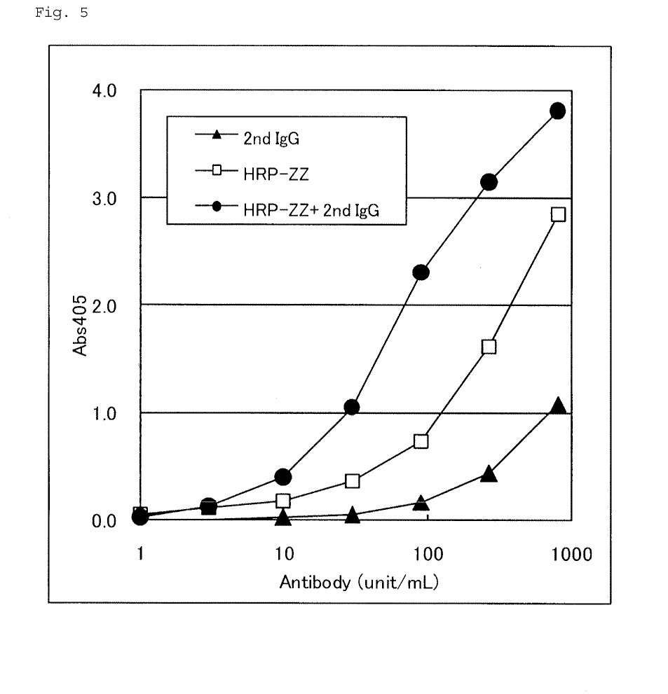

An experiment was carried out in the same manner as in Example 17 to investigate the antibody detection ELISA using HRP-labeled BNC-ZZ. The recombinant protein of Leishmania protozoa and control human antiserum used in this experiment were both obtained from Aichi Medical University, Department of Infection and Immunology. A recombination protein of the protozoa, which is a pathogen of Leishmania, was immobilized in place of Pre-s2 on an ELISA plate, and the plate was blocked with k-Block-e. The control human antiserum was added at various concentrations. Subsequently, an HRP-labeled anti-human IgG antibody dissolved in PBS-T containing Pluronic F-127 at a final concentration of 0.05%, an HRP-labeled BNC-ZZ obtained in Production Example 3, or a combination of the HRP-labeled anti-human IgG antibody and HRP-labeled BNC-ZZ at the same concentration were added, and a reaction was allowed to proceed. After washing, absorbance at 405 nm was measured using ABTS (1-STEP ABTS, produced by Thermo Scientific) as a substrate. FIG. 5 shows the results. The antibody titer measurement values are expressed in Unit/mL.

The results show that the use of HRP-labeled BNC-ZZ as indicated by “HRP-ZZ” with white squares in FIG. 5 achieves a slightly less than 10-fold higher sensitivity, compared to detection using the anti-human IgG antibody as indicated by “2nd IgG” with black triangles in FIG. 5, and that the combined use of HRP-ZZ and IgG as indicated by “HRP-ZZ+IgG” with black circles in FIG. 5 achieves an approximately 30-fold higher sensitivity.

The above results of Examples 17 and 18 show that HRP-labeled BNC-ZZ is useful for high-sensitivity detection in antibody detection ELISA and that, in particular, detection in the presence of a detection antibody enables higher sensitivity detection.

### Example 19

An experiment was carried out to investigate practical ELISA measurement using HRP-labeled BNC-ZZ. A GFP protein was immobilized on an ELISA plate and the plate was blocked with k-Block-e. An anti-GFP mouse IgG antibody of a known concentration was used as a standard. A 100-fold or more dilution of mouse anti-GFP antiserum was used as a sample. The standard and sample were individually added to wells of the ELISA plate. Subsequently, HRP-labeled BNC-ZZ of Production Example 3 dissolved in PBS containing Pluronic F-127 at a final concentration of 0.05%, a mixed complex of a rabbit-derived anti-IgG antibody and HRP-labeled BNC-ZZ, or a rabbit-derived HRP-labeled anti-mouse IgG antibody was added, and a reaction was allowed to proceed. After washing, absorbance at 450 nm was measured in the same manner as in Example 9. The measurement values were obtained by calculating the concentration of anti-GFP mouse IgG in the antiserum as mean±std (ng/mL, n=3), based on a calibration curve prepared by using the standard antibody.

As a result, the values obtained using HRP-labeled BNC-ZZ, and a mixed complex of HRP-labeled BNC-ZZ and anti-mouse IgG were 15613±936 and 15403±1192, respectively, and matched well with 15400±109 quantified by using an HRP-labeled anti-mouse IgG antibody, thus indicating that this measurement is a sufficiently quantitative analysis.

### Example 20

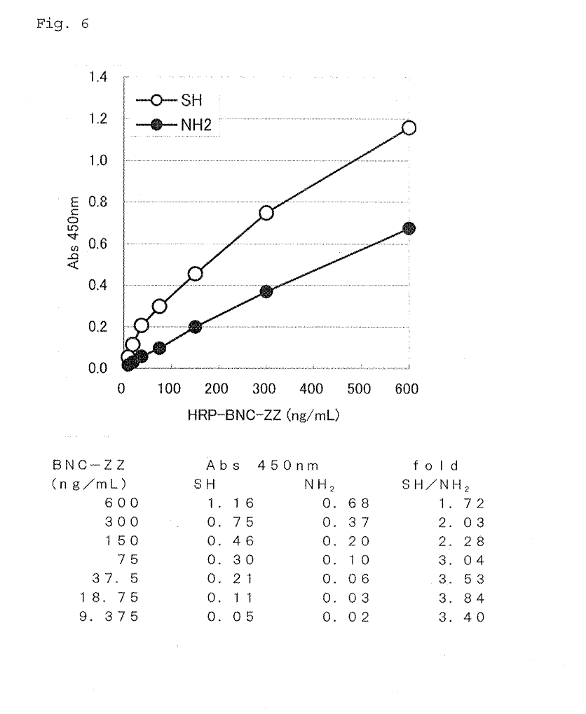

An experiment was carried out to investigate the antibody binding activity of ALP-labeled BNC-ZZ. A control rabbit-derived IgG was immobilized on an ELISA plate, and the plate was blocked with 1% Blockace. 50 μL each of NH₂-ALP-labeled BNC-ZZ or SH-ALP-labeled BNC-ZZ obtained in Production Example 4 were added at concentrations of 0, 9.375, 18.75, 37.5, 75, 150, 300, and 600 ng/mL in terms of the amount of BNC-ZZ protein to wells of the plate. After a reaction was allowed to proceed, the plate was washed and absorbance at 405 nm was measured in the same manner as in Example 2. Pluronic F-127 at a final concentration of 0.05% was used with ALP-labeled BNC-ZZ. FIG. 6 shows the results.

In all of the BNC-ZZ concentrations, measurement values obtained using SH-ALP-labeled BNC-ZZ were higher than those obtained using NH₂-ALP-labeled BNC-ZZ. At the concentration of 600 ng/mL, the measurement value obtained using SH-ALP-labeled BNC-ZZ was about 1.7 times higher than that of NH₂-ALP-labeled BNC-ZZ. This multiplication factor is almost the same as the level of difference therebetween in ALP specific activity (since the former has an ALP specific activity of 5.61 unit/μg and the latter has an ALP specific activity of 3.56 unit/μg, the former is 1.6 times higher). The results suggest both of SH-ALP-labeled BNC-ZZ and NH₂-ALP-labeled BNC-ZZ were bound substantially in the maximum amount to the immobilized antibody at these concentrations. In contrast, when the concentration is lower than this level, the multiplication factor of the measurement value obtained using SH-ALP-labeled BNC-ZZ relative to the value obtained using NH₂-ALP-labeled BNC-ZZ increased. When the concentration of the BNC-ZZ was 37.5 ng or less, the measurement value obtained using SH-ALP-labeled BNC-ZZ was about 3.6 times higher on the average.

This indicates that in low concentration regions, SH-ALP-labeled BNC-ZZ was bound to the immobilized antibody in a larger amount. Considering the fact that the difference in HRP specific activity was about 1.6 times, the results suggest that the binding affinity of SH-ALP-labeled BNC-ZZ for the antibody is about 2.3 times higher than that of NH₂-ALP-labeled BNC-ZZ. Labeling via NH₂ groups had a lower antibody binding capacity than labeling via SH groups probably for the same reason as for the HRP-labeling described above in Example 16.

### Example 21

An experiment was carried out to investigate the application of ALP-labeled BNC-ZZ to ELISA. A control mouse-derived IgG (polyclonal) was immobilized on an ELISA plate, and the plate was blocked using 0.5% casein. SH-ALP-labeled BNC-ZZ obtained in Production Example 4 or ALP-labeled rabbit-derived anti-mouse IgG antibody was added at various concentrations, and a reaction was allowed to proceed. After washing, absorbance at 405 nm was measured in the same manner as in Example 2.

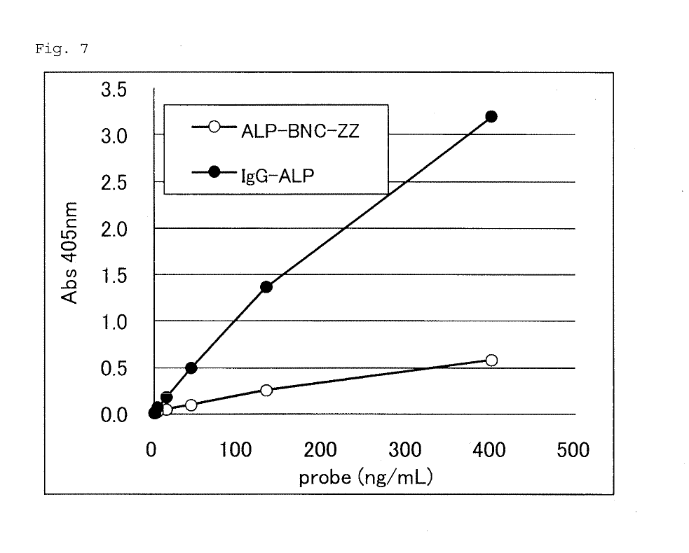

Pluronic F-127 at a final concentration of 0.05% was used with ALP-labeled BNC-ZZ. FIG. 7 shows the results.

The use of ALP-labeled BNC-ZZ achieved a much higher reaction than the use of ALP-labeled antibody. The results suggest that ALP-labeled BNC-ZZ is useful for high-sensitivity antibody detection.

### Example 22

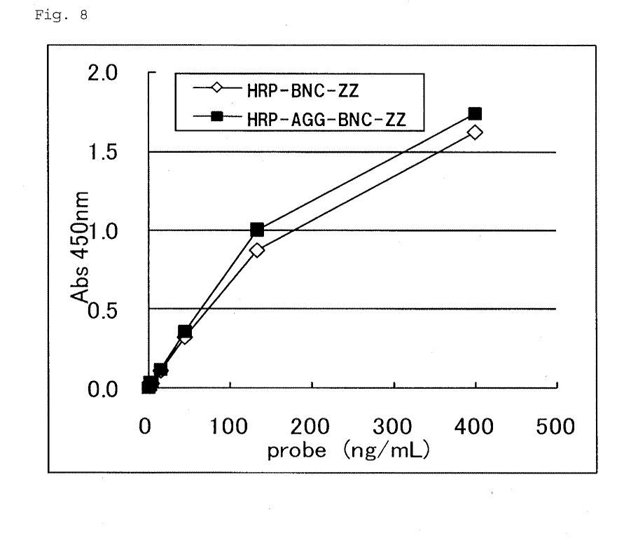

An experiment was carried out to evaluate the antibody binding activity of HRP-labeled AGG-BNC-ZZ. A control porcine-derived IgG (produced by our company using the method of Example 5) was immobilized on an ELISA plate and the plate was then blocked with 0.5% casein. As a probe, HRP-labeled AGG-BNC-ZZ of Production Example 6 or NH₂-HRP-labeled BNC-ZZ of Production Example 3 at various concentrations plotted on the abscissa of the graph shown in FIG. 8 was added to wells of the plate, and a reaction was allowed to proceed. After washing, absorbance at 450 nm was measured in the same manner as in Example 9. Pluronic F-127 was added to the probes at a final concentration of 0.05%. FIG. 8 shows the results.

As shown in Example 3, the HRP activity of HRP-labeled AGG-BNC-ZZ is known to be 1/3 of that of HRP-labeled BNC-ZZ. Supposing that both probes have the same antibody binding activity, the reaction of HRP-labeled AGG-BNC-ZZ with immobilized porcine-derived IgG would be expected to be 1/3 of that of HRP-labeled BNC-ZZ.

However, in reality, the reaction of HRP-labeled AGG-BNC-ZZ was about 1/1.1 to 1/2.4 of that of HRP-labeled BNC-ZZ. This indicates that HRP-labeled AGG-BNC-ZZ has higher antibody binding activity than HRP-labeled BNC-ZZ. The above results show that both of HRP-labeled AGG-BNC-ZZ and HRP-labeled BNC-ZZ have higher antibody binding activity than the antibody.

### Example 23

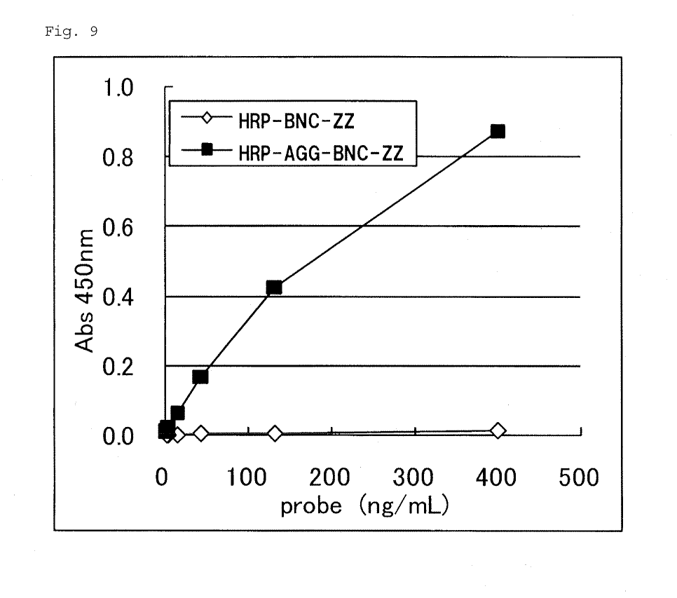

An experiment was carried out to evaluate the protein G-derived antibody binding capacity of HRP-labeled AGG-BNC-ZZ. The experiment was carried out in the same manner as in Example 22 except that a mouse-derived IgG₁, to which BNC-ZZ was considered to be difficult to bind, was immobilized in place of the control porcine-derived IgG used in Example 22. FIG. 9 shows the results.

The HRP-labeled AGG-BNC-ZZ exhibited a high binding reaction with mouse IgG₁. In contrast, substantially no binding reaction of HRP-labeled BNC-ZZ was detected. This indicates that the protein G-derived antibody binding sites of HRP-labeled AGG-BNC-ZZ functioned well, and HRP-labeled AGG-BNC-ZZ has higher binding capacity to antibodies to which protein A-derived antibody binding sites are difficult to bind.

### Example 24

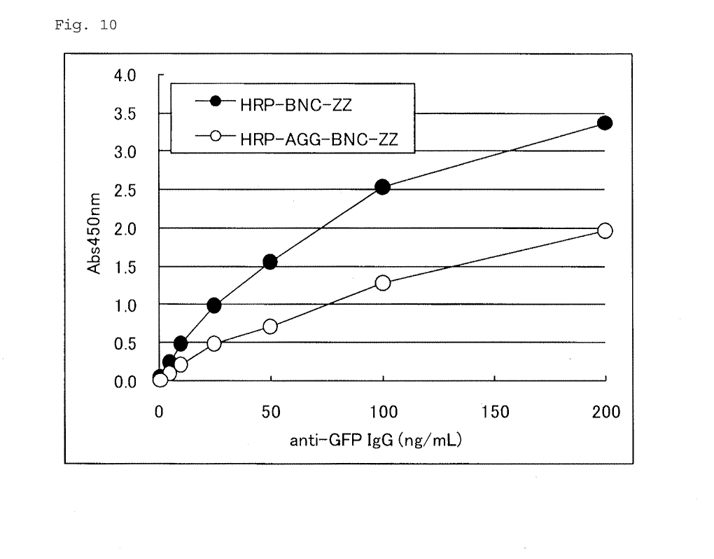

An experiment was carried out to investigate the application of HRP-labeled AGG-BNC-ZZ to ELISA. The GFP protein produced using E. coli was immobilized on an ELISA plate. Subsequently, the plate was blocked with k-Block-e. The rabbit-derived anti-GFP antibody was added to wells of the plate at various concentrations plotted on the abscissa of the graph shown in FIG. 10. 100 ng/mL of either HRP-labeled AGG-BNC-ZZ obtained in Production Example 6 or SH-HRP-labeled BNC-ZZ obtained in Production Example 3 was added as a probe, and a reaction was allowed to proceed. After washing, absorbance at 450 nm was measured in the same manner as in Example 9. Pluronic F-127 was added to each probe at a final concentration of 0.05%. FIG. 10 shows the results.

Similar to SH-HRP-labeled BNC-ZZ used as a control, HRP-labeled AGG-BNC-ZZ exhibited a reaction depending on the concentration of the antibody added. The reaction of HRP-labeled AGG-BNC-ZZ was about 1/2 of that of SH-HRP-labeled BNC-ZZ. Considering the fact that the HRP activity of HRP-labeled AGG-BNC-ZZ is 1/3 of that of HRP-labeled BNC-ZZ, HRP-labeled AGG-BNC-ZZ was found to exhibit a reaction higher than HRP-labeled BNC-ZZ.

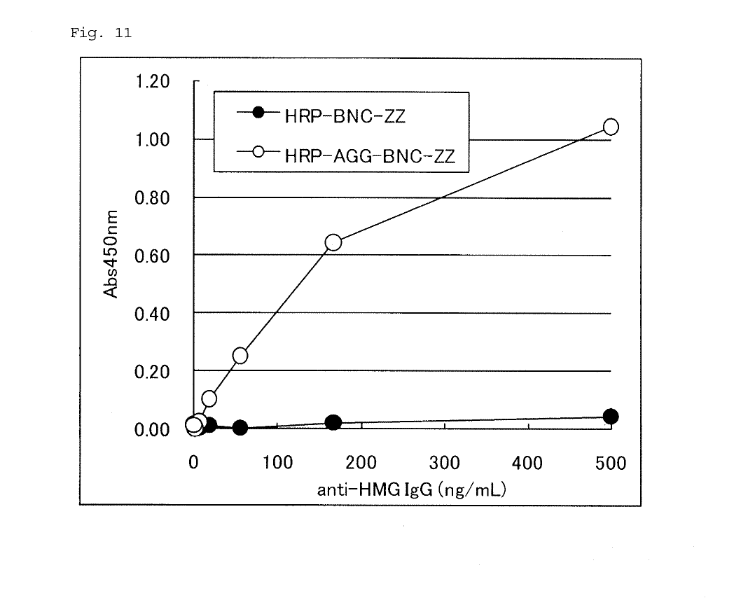

Subsequently, an experiment was carried out in the same manner as above except that a mouse-derived anti-HMG1 monoclonal antibody (Cosmobio; IgG₁) was immobilized in place of the rabbit-derived anti-GFP antibody on the ELISA plate. FIG. 11 shows the results.

When HRP-labeled AGG-BNC-ZZ was used as a detection probe, a reaction dependent on the concentration of the antibody used was observed. In contrast, when HRP-labeled BNC-ZZ was used, substantially no reaction was observed.

The above results show that HRP-labeled AGG-BNC-ZZ can be used in a practical measurement system of antibody detection ELISA. The results also show that HRP-labeled AGG-BNC-ZZ can detect mouse IgG₁ that cannot be detected with HRP-labeled BNC-ZZ and that HRP-labeled AGG-BNC-ZZ is highly useful.

### Example 25

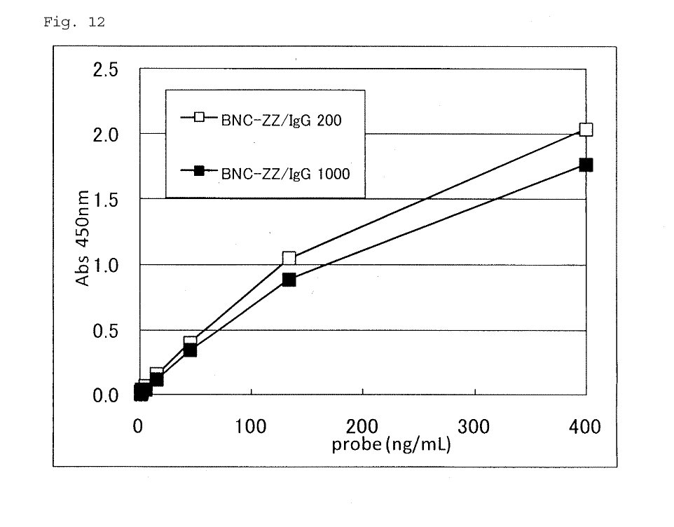

An experiment was carried out to investigate the antibody binding activity of HRP-labeled BNC-ZZ/rabbit-derived anti-mouse IgG antibody complexes. A control mouse-derived IgG was immobilized on an ELISA plate and then blocked by reaction with k-Block-e for 1 hour. Among the HRP-labeled BNC-ZZ/rabbit-derived anti-mouse IgG antibody complexes produced using a crosslinking agent BS₃ in Production Example 7, those produced using the crosslinking agent BS₃ at concentrations of 200 μM and 1,000 μM were added as probes at concentrations of 0, 0.55, 1.65, 4.94, 14.8, 44.4, 133, and 400 ng/mL, and a reaction was allowed to proceed. After washing, absorbance at 450 nm was measured in the same manner as in Example 9. Pluronic F-127 was added at a final concentration of 0.05%. FIG. 12 shows the results.

Reactions dependent on the concentrations of the complexes used were observed. A comparison of the complex obtained by using the 200 μM crosslinking agent and the complex obtained by using the 1,000 μM crosslinking agent shows that the latter complex had a slightly lower binding activity but both of the complexes exhibited practical levels of antibody binding activity.

### Example 26

An experiment was carried out to confirm whether anti-OVA mouse IgE and anti-OVA mouse IgG present in the anti-OVA mouse antiserum obtained by immunization with ovalbumin (OVA) can be practically measured. OVA was immobilized on ELISA plates and the plates were then blocked with h-Block-e. An anti-OVA mouse antiserum diluted 100 times or more was added to the plates. As probes for IgE assay, a complex of SH-HRP-labeled BNC-ZZ of Production Example 3 and anti-mouse IgE (produced by Nordic Immunology Lab) produced according to the method of Production Example 7 using BS₃ at a concentration of 1,000 μM, and HRP-labeled anti-mouse IgE were used. As a probe for IgG assay, a mixed complex of the SH-HRP-labeled BNC-ZZ of Production Example 3 and rabbit-derived anti-mouse IgG antibody was used. These probes were added to wells of the plates prepared for IgE and IgG assays and a reaction was allowed to proceed. After washing, absorbance at 450 nm was measured in the same manner as in Example 9. A calibration curve was prepared using a standard antibody. Based on the antibody titer of the standard antibody, the antibody titer of each antibody was calculated as mean±std (unit/mL, n=3).

When the HRP-labeled BNC-ZZ/anti-mouse IgE complex was used, the antibody titer of anti-OVAIgE in mouse serum was 3807±1439 nunit/mL. When the HRP-labeled anti-mouse IgE was used, the antibody titer of anti-OVAIgE in mouse serum was 3558±935 nunit/mL. Both of the titers were similar.

The antibody titer of the anti-OVA mouse IgG was 4175±8717 μunit/mL. The results show that the assay systems using these complexes enable sufficient measurement of antibody titers of IgE and IgG and are practically useful.

### Example 27

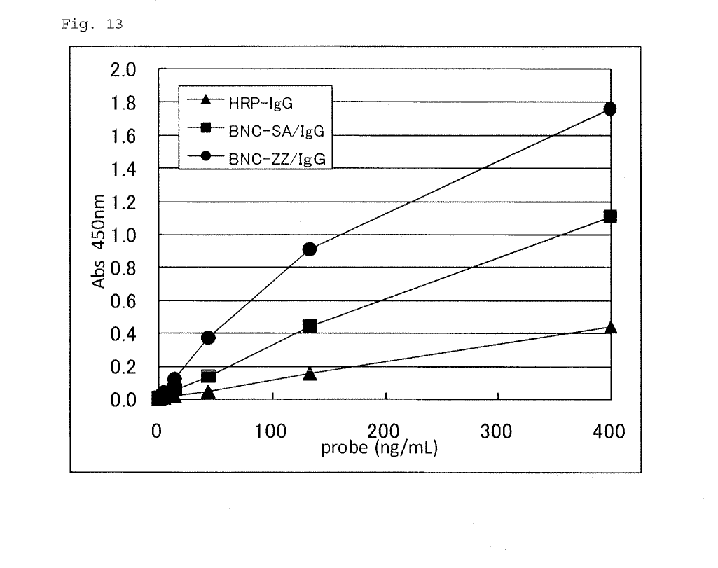

Using the HRP-labeled BNC-SA/rabbit-derived anti-mouse IgG antibody complex prepared in Production Example 8, an experiment was carried out to confirm the binding reaction with mouse IgG. A control mouse-derived IgG was immobilized on an ELISA plate, and the plate was then blocked with 1% Blockace. As a probe, the HRP-labeled BNC-SA/rabbit-derived anti-mouse IgG antibody complex obtained in Production Example 8 was used. As control probes, a mixed complex of NH₂-HRP-labeled BNC-ZZ of Production Example 3 and anti-mouse IgG rabbit antibody (produced by Bethyl) and a rabbit-derived HRP-labeled anti-mouse IgG antibody were used. These probes were individually added to wells of the plate at concentrations of 0, 0.55, 1.65, 4.94, 14.8, 44.4, 133, and 400 ng/mL. After washing, absorbance at 450 nm was measured in the same manner as in Example 9. Pluronic F-127 was added at a final concentration of 0.05%. FIG. 13 shows the results.

As the concentration of each probe increased, the reaction increased irrespective of the type of probe used. The binding activity of the HRP-labeled BNC-SA/antibody complex was about half of the binding activity of the mixed complex of the antibody and HRP-labeled BNC-ZZ that was labeled with HRP via the same NH₂ group. Considering the fact that the HRP enzyme activity of the former complex is about 1/3 of that of the latter complex, HRP-labeled BNC-SA/antibody complex exhibited a higher binding capacity to the antibody than the mixed complex of NH₂-HRP-labeled BNC-ZZ and antibody. On the other hand, the HRP-labeled BNC-SA/antibody complex exhibited a reaction that is about twice as high as that of the HRP-labeled antibody. The above results show that the HRP-labeled BNC-SA/antibody complex has a higher antibody binding capacity than HRP-labeled anti-mouse IgG antibody and is useful.

### Example 28

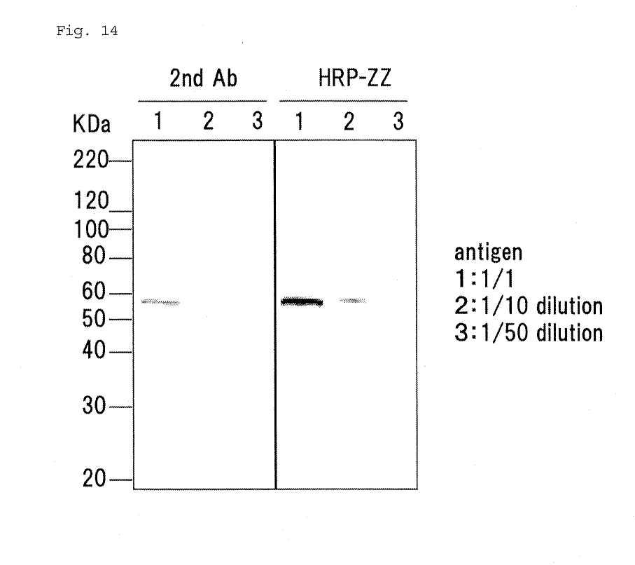

An extract of HuH7 cells was subjected to Western blotting in the same manner as in Example 13. The membrane was blocked with 5% skim milk, and a mouse-derived anti-Vimentin antibody (produced by Progen, 1/1,000 dilution) was used as a primary antibody. As a probe, a rabbit-derived HRP-labeled anti-mouse IgG antibody containing 1% skim milk (produced by Rockland, 1/10,000; 2nd An in FIG. 14) or HRP-labeled BNC-ZZ of Production Example 3 containing 0.1% Pluronic F-127 and 1% skim milk was used. FIG. 14 shows the results.

When detection was conducted using a rabbit-derived HRP-labeled anti-mouse IgG antibody as a probe, no band signal could be detected from a 1/10 dilution of extract. In contrast, when HRP-labeled BNC-ZZ was used, an equivalent signal was obtained even from a 1/10 dilution of extract. HRP-labeled BNC-ZZ was thus found to be effective for high-sensitivity detection.

### Example 29

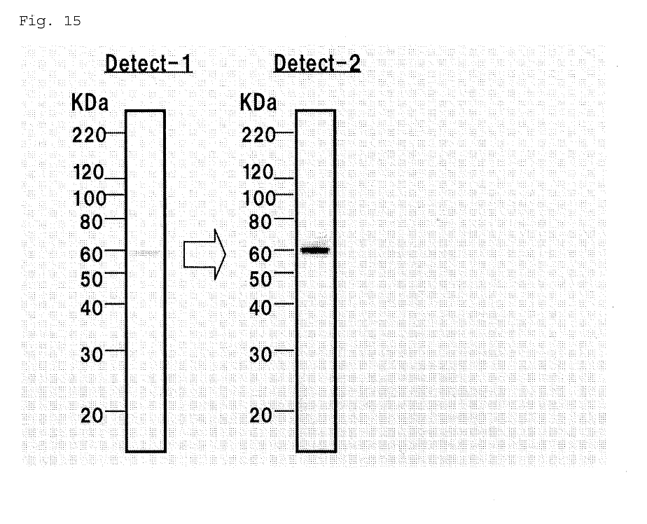

Western blotting was performed in the same manner as in Example 28. Detection was conducted using a 1/3,000 dilution of the anti-Vimentin mouse antibody as a primary antibody and using HRP-labeled anti-mouse IgG antibody (produced by Rockland, #611-1302, 1/10,000 dilution) as a secondary antibody (Detect-1 in FIG. 15). Further, after dissolution in a solution containing 0.1% Lipidure 802, detection was further conducted using HRP-labeled BNC-ZZ as an additional probe (Detect-2 in FIG. 15). FIG. 15 shows the results.

The results show that when poor detection sensitivity (Detection-1) was obtained by using the HRP-labeled anti-mouse antibody as a secondary antibody, redetection using HRP-labeled BNC-ZZ as an additional probe (Detection-2) was able to enhance the band signal. Although the data are not shown, the signal hardly increased even when the secondary antibody was added. Thus, since merely adding HRP-labeled BNC-ZZ can sensitize the signal, HRP-labeled BNC-ZZ is highly useful.

### Example 30

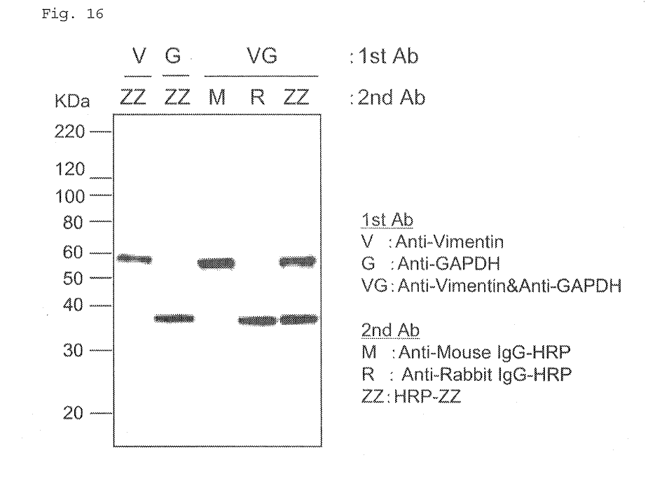

Western blotting was performed in the same manner as in Example 28. Detection was conducted using an anti-Vimentin mouse antibody (produced by Progen, 1/2,000 dilution), an anti-GAPDH rabbit antibody (produced by Epitomics, Inc., 1/10,000 dilution), or both of the antibodies as primary antibodies, and using an HRP-labeled anti-mouse IgG antibody (produced by Rockland), an HRP-labeled anti-rabbit IgG antibody (produced by Santa Cruz Biotechnology, Inc.), or an HRP-labeled BNC-ZZ (HRP-ZZ) as a secondary antibody. HRP-labeled BNC-ZZ was used with Pluronic F-127 that was added at a final concentration of 0.1%. FIG. 16 shows the results.

The results show that when HRP-labeled BNC-ZZ is used, Vimentin and GAPDH can be detected at one time at the positions detected with the respective secondary antibodies. The results further show that when HRP-labeled BNC-ZZ is used, Vimentin and GAPDH can be equally detected simultaneously even when the antibodies are derived from different animal species.

### Example 31

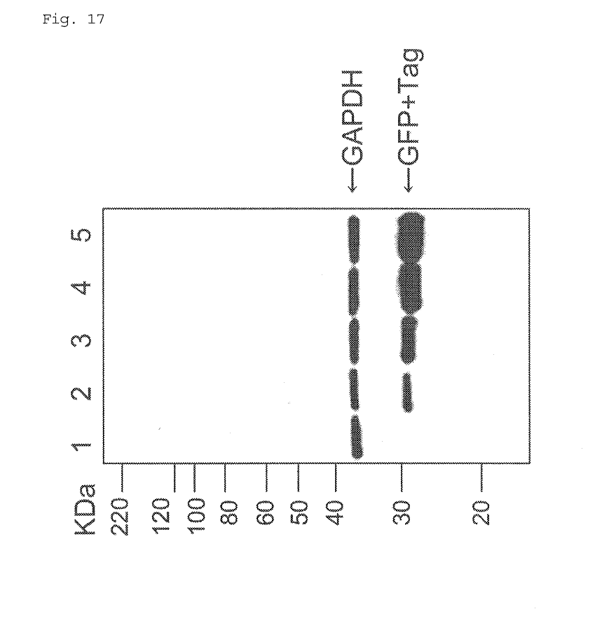

As a sample, a two-fold dilution series of GFP-Histag protein was added to an HuH7 cell extract, and the resulting mixture was subjected to Western blotting in the same manner as in Example 28. The resulting product was reacted with an anti-GAPDH rabbit antibody (produced by Epitomics, Inc., 1/10,000) and an anti-GFP rabbit antibody (produced by Rockland, 1/2,000) as primary antibodies. GAPDH and GFP were then simultaneously detected using HRP-labeled BNC-ZZ (HRP-ZZ) diluted with PBS-T containing 0.1% Lipidure 206. FIG. 17 shows the results.

When HRP-labeled BNC-ZZ was used, signals increased as the concentration of GFP protein increased. This was thus found to be a quantitative assay.

### Example 32

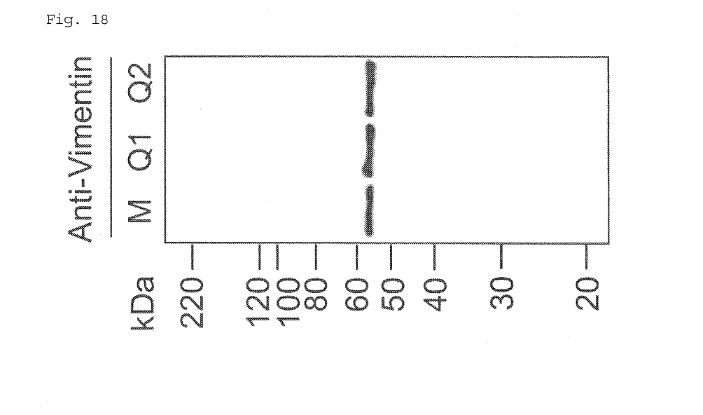

Western blotting was performed in the same manner as in Example 28. An anti-Vimentin mouse antibody (produced by Progen, 1/2,000) and HRP-labeled BNC-ZZ were mixed beforehand in equal amounts and the mixed complex was added to a PVDF membrane to perform detection using a one-step method. TBS-T containing 0.1% Pluronic F-127 was used as a reaction solution of the mixed complex of the HRP-labeled BNC-ZZ and the antibody. As a control, detection was also performed by a two-step method. Specifically, after a reaction with an anti-Vimentin mouse antibody was performed, a reaction with an HRP-labeled anti-mouse antibody (produced by Rockland, 1/5,000) was performed. FIG. 18 shows the results. The one-step method took a total of about 65 minutes to perform the following operations in the following order after the transfer of the protein to the PVDF membrane until the detection: blocking for 5 minutes (Q1 in FIG. 18) or 15 minutes (Q2 in FIG. 18), washing for 5 minutes, a reaction (using primary antibody+HRP-labeled BNC-ZZ) for 30 minutes, and washing for 25 minutes (5 minutes×5: Q1) or 15 minutes (3 minutes×5: Q2). In contrast, the two-step method using an HRP-labeled anti-mouse antibody (M in FIG. 18) took a total of about 230 minutes to perform the following operations in the following order: blocking for 60 minutes, washing for 10 minutes (5 minutes×2), a primary antibody reaction for 60 minutes, washing for 15 minutes (5 minutes×3), a secondary reaction for 60 minutes, and washing for 25 minutes (5 minutes×5).

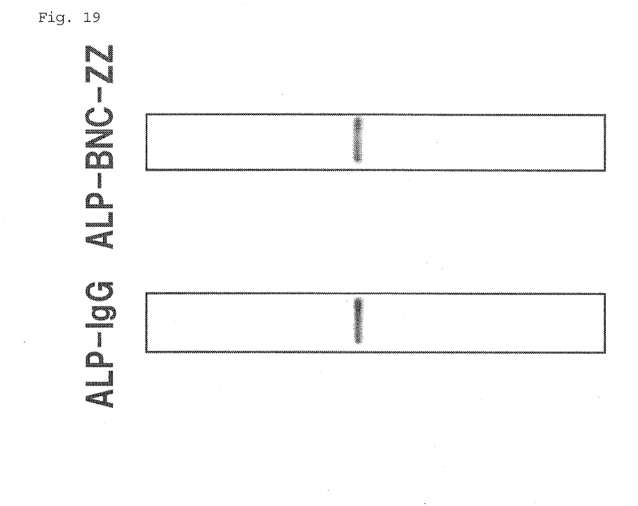

The usual two-step method required a total of 230 minutes of operations until signals were detected using a protein transcription membrane. In contrast, the one-step method using HRP-labeled BNC-ZZ (Q1 and Q2 in FIG. 19) was able to shorten the washing time and the times for other operations to thereby reduce the required time to a total of 65 minutes while achieving equivalent results. HRP-labeled BNC-ZZ was thus found to be useful for enabling rapid detection Western blotting.

### Example 33

Western blotting was performed in the same manner as in Example 28. Anti-p53 rabbit antibody (produced by Santa Cruz, 1/200) was used as a primary antibody. ALP labeling anti-rabbit IgG antibody (produced by Sigma, 1/50,000) or SH-ALP-labeled BNC-ZZ obtained in Production Example 4 was used as a secondary antibody. CDP-Star (produced by NEB) was used as a substrate for ALP. FIG. 19 shows the results.

Approximately similar signals were obtained by detection using the ALP labeling anti-rabbit IgG antibody and detection using ALP-labeled BNC-ZZ. The results suggest that ALP-labeled BNC-ZZ is also highly useful as a probe in Western blotting and is applicable to various methods of use as described in the Examples of this application using HRP-labeled BNC-ZZ.

### Example 34

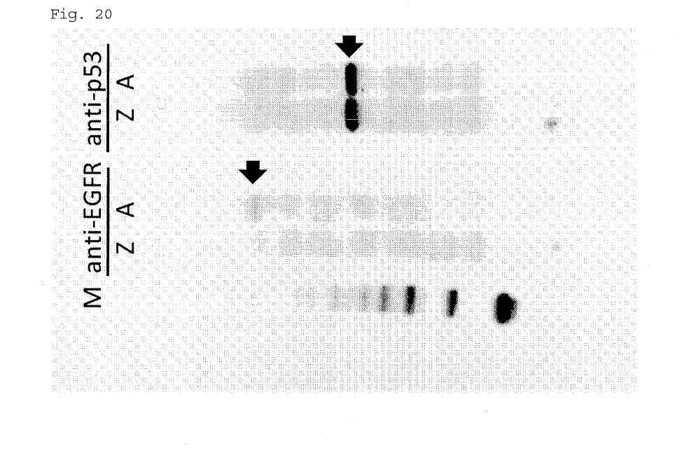

Western blotting was performed in the same manner as in Example 28 except that an A431 cell extract was used instead. The extract was reacted with an anti-EGFR antibody (produced by Cell Signaling, 1/1,000 dilution) whose species is mouse IgG₁, or an anti-p53 rabbit antibody (produced by Santa Cruz, 1/200 dilution) as a primary antibody, and then reacted with HRP-labeled AGG-BNC-ZZ of Production Example 6 or HRP-labeled BNC-ZZ of Production Example 3 as diluted with TBS-T containing 0.1% Pluronic F-127. FIG. 20 shows the results.

When an anti-EGFR antibody, which is mouse IgG₁, was used, the antibody was not detected with HRP-labeled BNC-ZZ at all (Z in FIG. 20). In contrast, when HRP-labeled AGG-BNC-ZZ (A in FIG. 20) was used, the antibody was detectable. When an anti-p53 antibody, which is rabbit IgG, was used, the antibody was detected well whether HRP-labeled BNC-ZZ or HRP-labeled AGG-BNC-ZZ was used.

The above results match well with the fact that HRP-labeled AGG-BNC-ZZ, which has protein G-derived antibody binding sites, can also easily bind to mouse IgG₁, whereas HRP-labeled BNC-ZZ, which has only protein A-derived antibody binding sites, hardly binds to mouse IgG₁. The results prove that HRP-labeled AGG-BNC-ZZ is useful.

### Production Example 9

### Preparation of HRP-Labeled BNC-L

HRP-labeled BNC-L was obtained by labeling BMC-L of Production Example 1 with HRP via SH using the Peroxidase Labeling Kit-SH.

### Example 35

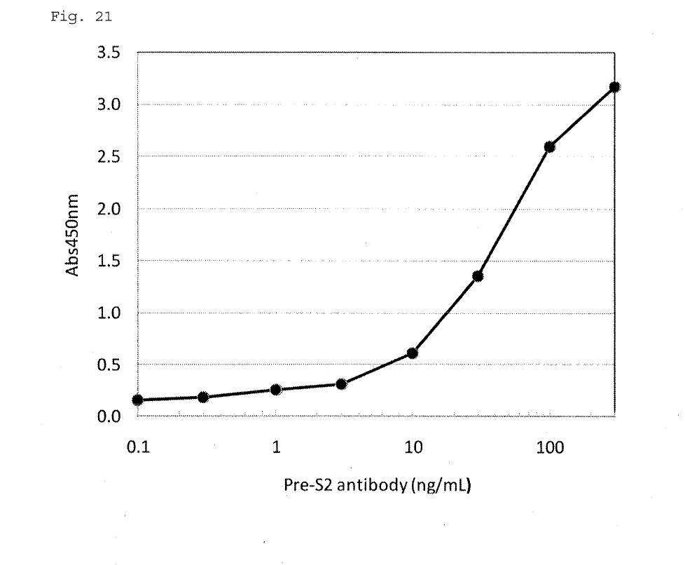

Pre-S2, which is a peptide of hepatitis B virus surface antigen, was immobilized on an ELISA plate, and the plate was blocked. Anti-Pre-52 antibody was added at various concentrations to wells of the plate. Subsequently, HRP-labeled BNC-L obtained in Production Example 9 was added and a reaction was allowed to proceed. After washing, absorbance at 450 nm was measured in the same manner as in Example 9 (antigen sandwich ELISA). FIG. 21 shows the results.

The results clearly show that HRP-labeled BNC-L is sufficiently usable as a probe for an ELISA assay.

### Production Example 10

### SH-HRP-Labeled BNC-(Sugar Chain)-AGG

BNC-L obtained in Production Example 1 was subjected to oxidative treatment with NaIO₄ to form an aldehyde group at a sugar residue in a sugar chain added to BNC-L. Subsequently, the resulting product was reacted with an AGG protein to bind the aldehyde group to a lysine residue of the AGG peptide. After a NaBH₄ solution was added to the reaction mixture, the mixture was subjected to gel filtration to obtain BNC-AGG. Further, using the Peroxidase Labeling Kit-SH, the obtained BNC-AGG was labeled with HRP via SH of BNC-AGG. Specifically, the obtained ENC has AGG bound thereto via its sugar chain and also has HRP bound thereto via its SH (hereinafter sometimes referred to as “SH-HRP-labeled BNC-(sugar chain)-AGG”).

### Example 36

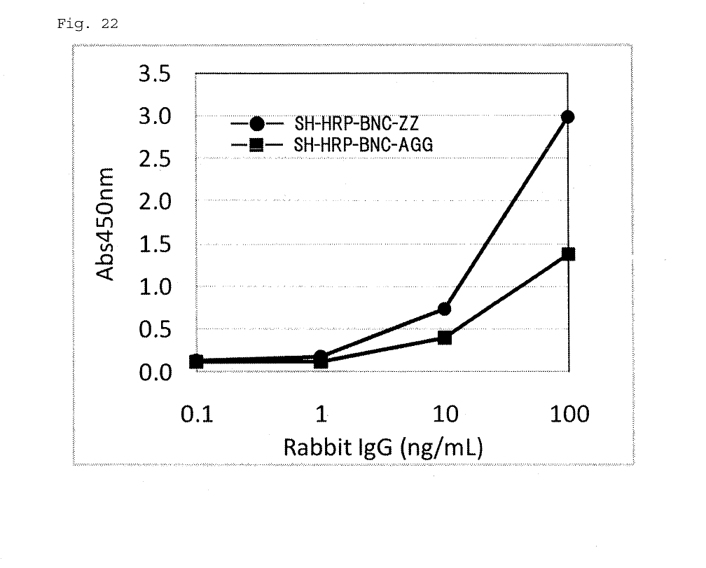

Rabbit IgG at various concentrations was immobilized on an ELISA plate and the plate was blocked. SH-HRP-labeled BNC-(sugar chain)-AGG obtained in Production Example 10 was added to wells of the plate, and a reaction was allowed to proceed. After washing, absorbance at 450 nm was measured in the same manner as in Example 9. FIG. 22 shows the results.

The results show that although the absorbance at 450 nm obtained using SH-HRP-labeled BNC-(sugar chain)-AGG is lower than that obtained using SH-HRP-labeled ZZ of Production Example 4 as a control, SH-HRP-labeled BNC-(sugar chain)-AGO has sufficient detection capability.

### Production Example 11

### Preparation of HRP-Labeled HVJ-E

Using the Peroxidase Labeling Kit-SH, HVJ-E (Genome One, produced by Ishihara Sangyo Kaisha, Ltd.), which is a virus-like particle comprising an envelope protein of Sendai Virus, was labeled with HRP via SH of cysteine residue of the protein present on the particle surface of HVJ-E to obtain an HRP-labeled HVJ-E.

### Example 37

The HRP activity of HRP-labeled HVJ-E obtained in Production Example 11 was measured in the same manner as in Example 1. The HRP activity was found to be 0.05 U/μg.

Virus-like particles having a transmembrane protein often have SH in or near the transmembrane region of the protein. This Example demonstrates that HVJ-E can also be labeled via SH and that SH groups present in virus-like particles are useful as' a target for labeling.

1. A virus-like particle for an immunoassay comprising a protein having self-organization ability, the protein having self-organization ability being an HBsAg protein, and the particle being modified with a biologically active molecule at at least one cysteine residue of the protein via a thiol group or thiol groups thereof.

2. (canceled)

3. The virus-like particle according to claim 1 wherein the protein having self-organization ability comprises an amino acid sequence of SEQ ID NO: 1.

4. The virus-like particle according to claim 1, wherein the protein having self-organization ability has an antibody-binding domain.

5. The virus-like particle according to claim 1, wherein the biologically active molecule is at least one member selected from the group consisting of an enzyme, an antibody-binding domain, biotin, a fluorescent dye, a luminescent dye, and an avidin compound.

6. The virus-like particle according to claim 5, wherein the antibody-binding domain is at least one member selected from the group consisting of antibody-binding domains of protein A, antibody-binding domains of protein G, and antibody-binding domains of protein L.

7. The viral particle according to claim 4, wherein the antibody-binding domain consists of an amino acid sequence of any one of SEQ ID NOS: 3 to 5.

8. The virus-like particle according to claim 1, wherein the biologically active molecule is an antibody-binding domain, and wherein an antibody is bound to the antibody-binding domain.

9. A blocking agent for an immunoassay using the virus-like particle according to claim 1, the blocking agent containing at least one member selected from the group consisting of hydroxyalkyl cellulose, polyvinyl alcohol, an ethylene oxide-propylene oxide copolymer, and a copolymer of 2-methacryloyloxyethylphosphocholine.

10. The blocking agent according to claim 9, wherein the ethylene oxide-propylene oxide copolymer is Pluronic®.

11. The blocking agent according to claim 9, wherein the ethylene oxide-propylene oxide copolymer is Pluronic® F127 and/or Pluronic® P105.

12. A kit for an immunoassay comprising the virus-like particle according to claim 1 and a blocking agent containing at least one member selected from the group consisting of hydroxyalkyl cellulose, polyvinyl alcohol, an ethylene oxide-propylene oxide copolymer, and a copolymer of 2-methacryloyloxyethylphosphocholine.
